(19) United States
(12) Patent Application Publication
(10) Pub. No.: US 2007/0015766 A1
(43) Pub. Date: Jan. 18, 2007

(54) Quinoline Derivatives as NK-3 and NK-2 Antagonists

(75) Inventors: Carlo Farina, Baranzate di Bollate (IT); Stefania Gagliardi, Baranzate di Bollate (IT); Giuseppe Giardina, Baranzate di Bollate (IT); Mario Grugni, Baranzate di Bollate (IT); Marisa Martinelli, Baranzate di Bollate (IT); Guy Marguerite Marie Gerard Nadler, Rennes (FR)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignees: GlaxoSmithKline S.P.A. & Laboratoire; GlaxoSmithKline S.A.S.

(21) Appl. No.: 11/426,414

(22) Filed: Jun. 26, 2006

Related U.S. Application Data
(63) Continuation of application No. 10/416,596, filed on Oct. 23, 2003, now abandoned.

(30) Foreign Application Priority Data
Nov. 13, 2000 (GB) 0027696.4
Apr. 11, 2001 (GB) 0109119.8

(51) Int. Cl. A61K 31/496; C07D 403/02

(52) U.S. Cl. 514/253.06; 544/363

(57) ABSTRACT

Certain compounds of formula (I) below or a pharmaceutically acceptable salt or hydrate thereof: 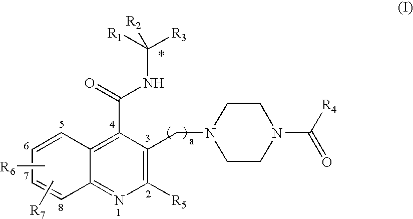 wherein:
R₁ is H or alkyl;
R₂ is aryl, cycloalkyl or heteroaryl;
R₃ is H or C₁₋₃ alkyl, optionally substituted by one or more fluorines;
R₄ is H, R₈NR₉R₁₀, R₁₁R₁₃ or R₁₁R₁₂R₁₃; or
R₅ is branched or linear alkyl, cycloalkyl, aryl, arylalkyl, or a single or fused ring aromatic heterocyclic group; a process for preparing such compounds, a pharmaceutical composition comprising such compounds and the use of such compounds and the use of such compounds and composition in medicine.

This application is a continuation of application Ser. No. 10/416,596, filed Oct. 23, 2003, which is a 371 of International Application No. PCT/EP01/13139, filed Nov. 12, 2001.

The present invention relates to novel compounds, in particular to novel quinoline derivatives, to processes for the preparation of such compounds, to pharmaceutical compositions containing such compounds and to the use of such compounds in medicine.

The mammalian peptide Neurokinin B (NKB) belongs to the Tachykinin (TK) peptide family which also include Substance P (SP) and Neurokinin A (NKA). Pharmacological and molecular biological evidence has shown the existence of three subtypes of TK receptor (NK₁, NK₂ and NK₃) and NKB binds preferentially to the NK₃ receptor although it also recognises the other two receptors with lower affinity (Maggi et al, 1993, J. Auton. Pharmacol., 13, 23-93).

Selective peptidic NK₃ receptor antagonists are known (Drapeau, 1990 Regul. Pept., 31, 125-135), and findings with peptidic NK₃ receptor agonists suggest that NKB, by activating the NK₃ receptor, has a key role in the modulation of neural input in airways, skin, spinal cord and nigro-striatal pathways (Myers and Undem, 1993, J. Physiol., 470, 665-679; Counture et al., 1993, Regul. Peptides, 46, 426-429; Mccarson and Krause, 1994, J. Neurosci., 14 (2), 712-720; Arenas et al. 1991, J. Neurosci., 11, 2332-8). However, the peptide-like nature of the known antagonists makes them likely to be too labile from a metabolic point of view to serve as practical therapeutic agents.

International Patent Application, Publication number WO 00/31037 discloses certain compounds stated to be non-peptide NK-3 antagonists and also to have NK-2 antagonist activity. These compounds are disclosed to be of potential use in the prevention and treatment of a wide variety of clinical conditions which are characterised by overstimulation of the Tachykinin receptors, in particular NK-3 and NK-2.

We have now discovered a further novel class of potent non-peptide NK-3 antagonists some of which fall within the generic scope of WO 00/31037. The new compounds are also far more stable from a metabolic point of view than the known peptidic NK-3 receptor antagonists and are of potential therapeutic utility. The new compounds also have good NK-2 antagonist activity and are therefore considered to be of potential use in the prevention and treatment of a wide variety of clinical conditions which are characterised by overstimulation of the Tachykinin receptors, in particular NK-3 and NK-2. The new compounds also show improved oral bioavailability.

These conditions include respiratory diseases, such as chronic obstructive pulmonary disease (COPD), asthma, airway hyper-reactivity, cough; inflammatory diseases such as inflammatory bowel disease, psoriasis, fibrositis, osteoarthritis, rheumatoid arthritis and inflammatory pain; neurogenic inflammation or peripheral neuropathy, allergies such as eczema and rhinitis; ophthalmic diseases such as ocular inflammation, conjunctivitis, vernal conjuctivitis and the like; cutaneous diseases, skin disorders and itch, such as cutaneous wheal and flare, contact dermatitis, atopic dermatitis, urticaria and other eczematoid dermatitis; adverse immunological reactions such as rejection of transplanted tissues and disorders related to immune enhancement or suppression such as systhemic lupus erythematosis; gastrointestinal (GI) disorders and diseases of the GI tract such as disorders associated with the neuronal control of viscera such as ulcerative colitis, Crohn's disease, irritable bowel syndrome (IBS), gastro-exophageous reflex disease (GERD); urinary incontinence and disorders of the bladder function; renal disorders; increased blood pressure, proteinuria, coagulopathy and peripheral and cerebral oedema following pre-eclampsia in pregnancies (hereinafter referred to as the ‘Primary Conditions’).

Certain of these compounds also show CNS activity and hence are considered to be of particular use in the treatment of disorders of the central nervous system such as anxiety, depression, psychosis and schizophrenia; neurodegenerative disorders such as AIDS related dementia, senile dementia of the Alzheimer type, Alzheimer's disease, Down's syndrome, Huntingdon's disease, Parkinson's disease, movement disorders and convulsive disorders (for example epilepsy); demyelinating diseases such as multiple sclerosis and amyotrophic lateral sclerosis and other neuropathological disorders such as diabetic neuropathy, AIDS related neuropathy, chemotherapy-induced neuropathy and neuralgia; addiction disorders such as alcoholism; stress related somatic disorders; reflex sympathetic dystrophy such as shoulder/hand syndrome; dysthymic disorders; eating disorders (such as food intake disease); fibrosing and collagen diseases such as scleroderma and eosinophilic fascioliasis; disorders of the blood flow caused by vasodilatation and vasospastic diseases such as angina, migraine and Reynaud's disease and pain or nociception, for example, that is attributable to or associated with any of the foregoing conditions especially the transmission of pain in migraine, (hereinafter referred to as the ‘Secondary Conditions’).

The compounds of formula (I) are also considered to be useful as diagnostic tools for assessing the degree to which neurokinin-3 and neurokinin-2 receptor activity (normal, overactivity or underactivity) is implicated in a patient's symptoms.

According to the present invention there is provided a compound of formula (I) below or a pharmaceutically acceptable salt or hydrate thereof: 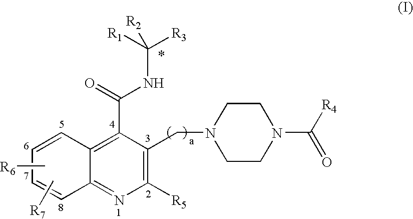
wherein:
R₁ is H or alkyl;
R₂ is aryl, cycloalkyl or heteroaryl;
R₃ is H or C₁₋₃ alkyl, optionally substituted by one or more fluorines;
R₄ is H, R₈NR₉R₁₀, R₁₁R₁₃ or R₁₁R₁₂R₁₃;
R₈ is a single bond or alkyl;
R₉ and R₁₀ are selected independently from H, alkyl, cycloalkyl or cycloalkylC₁₋₃alkyl, aryl or arylC₁₋₃alkyl, or R₉ and R₁₀ together with the nitrogen atom to which they are attached form a saturated or unsaturated heterocyclic ring which is optionally substituted by one or more fluorines;
R₁₁ is alkyl, alkenyl, aryl, heteroaryl, a saturated or unsaturated carbon ring including one or more heteroatoms selected from N, O and S, cycloalkyl, arylalkyl or cycloalkylalkyl, optionally substituted one or more times by C₁₋₃ alkyl, phenyl and/or phenylC₁₋₃ alkyl;
R₁₂ is alkyl or alkoxy, optionally substituted one or more times by C₁₋₃ alkyl and/or by phenyl;
R₁₃ is H or COO R₁₄;
R₁₄ is H or alkyl;
R₅ is branched or linear alkyl, cycloalkyl, cycloalkylalkyl, aryl, arylalkyl, or a single or fused ring aromatic heterocyclic group;
R₆ represents H or up to three substituents independently selected from the list consisting of: alkyl, alkenyl, aryl, alkoxy, hydroxy, halogen, nitro, cyano, carboxy, carboxamido, sulphonamido, alkoxycarbonyl, trifluoromethyl, acyloxy, amino or mono- or di-alkylamino;
R₇ is H or halo;
a is 1-6; and
any of R₂, R₅, R₈, R₉, R₁₀, R₁₁, R₁₂ and R₁₄ may optionally be substituted one or more times by halo, hydroxy, amino, cyano, nitro, carboxy or oxo;

subject to the proviso that said compound is not a compound of formula (I) wherein R₇ represents H, R₆ represents H, R₅ represents phenyl, and R₁, R₂, R₃, R₄, and a are one of the following combinations:

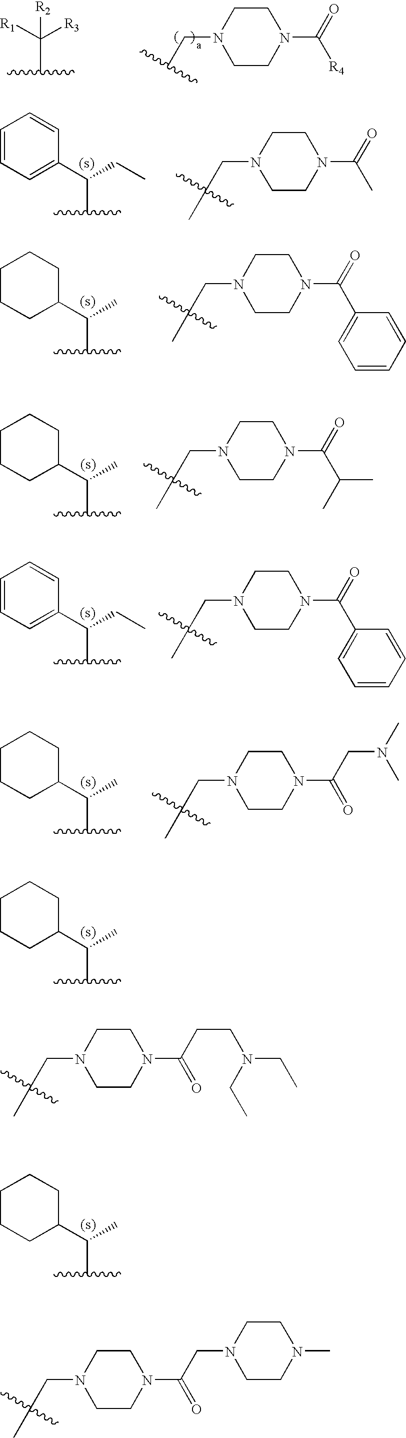

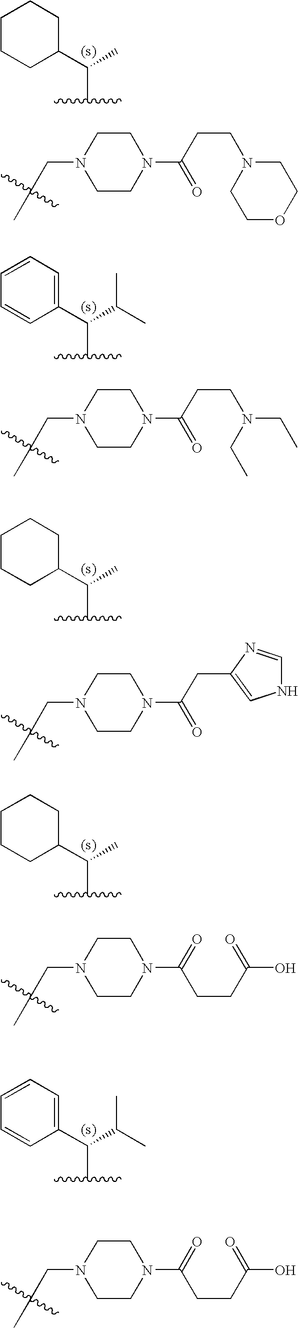

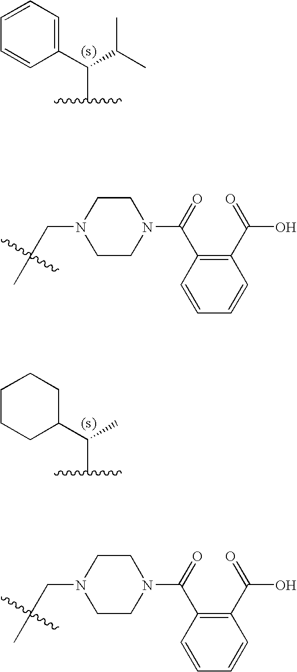

Advantageously, R₃ represents methyl, ethyl or isopropyl. Preferably, R₃ represents methyl.

Suitably, R₂ represents unsubstituted phenyl or unsubstituted cyclohexyl.

Preferably, R₁ is hydrogen.

Optionally, R₅ is unsubstituted phenyl.

Preferably, each of R₆ and R₇ represents hydrogen.

In preferred embodiments, a is 1, 2 or 3. In especially preferred embodiments, a is 1.

Advantageously, R₄ is H.

In some embodiments, R₄ is R₈NR₉R₁₀ and R₈ is a single bond, or methyl, or ethyl. Optionally, each of R₉ and R₁₀ may be H. Alternatively, one of R₉ and R₁₀ may be H, and the other of R₉ and R₁₀ may be methyl or ethyl or phenyl. Alternatively, R₉ and R₁₀ together with the N atom to which they are attached may form a saturated heterocyclic ring comprising exactly one N heteroatom.

Favourably R₄ is —CH₂CH₂NR₉R₁₀. In one aspect R₄ is —CH₂CH₂NR₉R₁₀ wherein R₉ and R₁₀ together with the nitrogen atom to which they are attached form a saturated or unsaturated heterocyclic ring as defined above, especially a saturated heterocyclic ring such as a pyrrolidine or piperidine ring.

In other embodiments, R₄ is R₁₁R₁₃ or R₁₁R₁₂R₁₃. R₁₁ may be a six-membered heteroaryl ring having one or two N heteroatoms, or a phenyl ring. Preferably, said heteroaryl or phenyl ring may be ortho-, para- or meta-linked to R₁₂ or R₁₃. Alternatively, R₁₁ may be cycloalkylalkyl, or alkyl substituted by alkyl or phenyl. Suitably, R₁₂ may be methyl or methoxy. Preferably, R₁₃ may be COO R₁₄, where R₁₄ is H or methyl or ethyl.

Suitably, R₄ is R₈NR₉R₁₀. Suitably, R₄ is R₁₁R₁₃. Suitably, R₄ is R₁₁R₁₂R₁₃.

When R₄ is a group —R₁₁COOR₁₄, R₁₄ is as defined in relation to fomula (I) and R₁₁ is a heteroaryl group, preferably the —COOR₁₄ group is attached to a carbon atom. In a particular aspect the atom, preferably a carbon atom, to which the —COOR₁₄ group is attached is spaced one or two atoms, suitably carbon atoms, from the point of attachment of R₄.

Suitably R₄ is a moiety of formula (a): 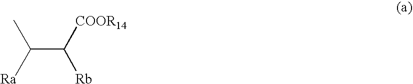
wherein R₁₄ is as defined in relation to formula (I) and Rₐ together with R_{b} represents a bond or Rₐ together with R_{b} and the carbon atoms to which they are attached represent cycloalkyl or heteroaryl.

In one aspect Rₐ together with R_{b} represents a bond. In one aspect Rₐ together with R_{b} and the carbon atoms to which they are attached represent cycloalkyl, such as cyclopropyl or cyclohexyl, or heteroaryl, such as pyrazine.

In particular compounds a is 1, R₆ is H, R₁ is H, R₅ is unsubstituted phenyl, R₇ is hydrogen, R₂ and R₃ are as defined above and R₄ is a moiety —R₁₁COOR₁₄, especially a moiety of formula (a).

In especially preferred embodiments, a is 1, R₆ is H, R₁ is H, R₅ is unsubstituted phenyl, R₇ is hydrogen, and R₂, R₃ and R₄ are selected from the following combinations:

| R₂ | R₃ | R₄ |
|---|---|---|
| Phenyl | ethyl | 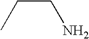 |
| Phenyl | methyl | 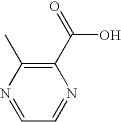 |
| Phenyl | methyl | 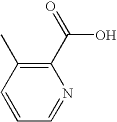 |
| Phenyl | methyl | 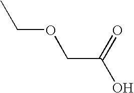 |
| Phenyl | methyl | 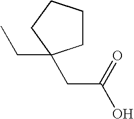 |
| Phenyl | methyl | 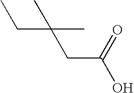 |
| Phenyl | methyl | 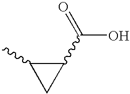 |
| Phenyl | methyl | 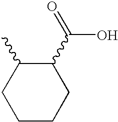 |
| Phenyl | methyl | 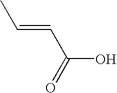 |
| Cyclohexyl | methyl | 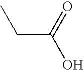 |
| Cyclohexyl | methyl | 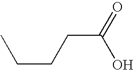 |
| Cyclohexyl | methyl | 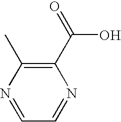 |
| Cyclohexyl | methyl | 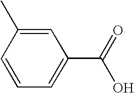 |
| Cyclohexyl | methyl | 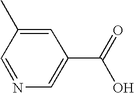 |
| Cyclohexyl | methyl | 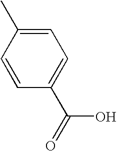 |
| Cyclohexyl | methyl | 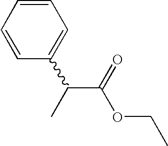 |
| Cyclohexyl | methyl | 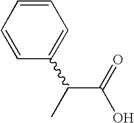 |
| Cyclohexyl | methyl | —H |
| Cyclohexyl | methyl | 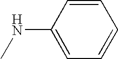 |
| Cyclohexyl | methyl | —NH₂ |
| cyclohexyl | methyl | 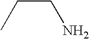 |
| cyclohexyl | methyl | 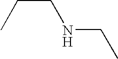 |
| cyclohexyl | methyl | 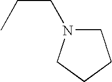 |
| cyclohexyl | methyl | 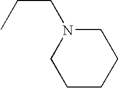 |

The compounds of formula (I) may have at least one asymmetric centre—for example the carbon atom labelled with an asterisk (*) in the compound of formula (I)—and therefore may exist in more than one stereoisomeric form. The invention extends to all such stereoisomeric forms and to mixtures thereof, including racemates. In particular, the invention includes compounds wherein the asterisked carbon atom in formula (I) has the stereochemistry shown in formula (Ia): 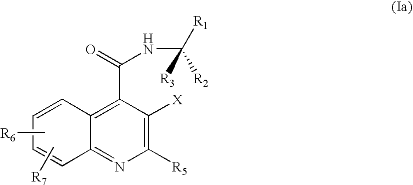
wherein R₁, R₂, R₃, R₅, R₆, and R₇ are as defined in relation to formula (I), and X represents the moiety

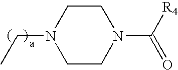

The compounds of formula (I) or their salts or solvates are preferably in pharmaceutically acceptable or substantially pure form. By pharmaceutically acceptable form is meant, inter alia, having a pharmaceutically acceptable level of purity excluding normal pharmaceutical additives such as diluents and carriers, and including no material considered toxic at normal dosage levels.

A substantially pure form will generally contain at least 50% (excluding normal pharmaceutical additives), preferably 75%, more preferably 90% and still more preferably 95% of the compound of formula (I) or its salt or solvate.

One preferred pharmaceutically acceptable form is the crystalline form, including such form in pharmaceutical composition. In the case of salts and solvates the additional ionic and solvent moieties must also be non-toxic.

Suitable salts are pharmaceutically acceptable salts.

Suitable pharmaceutically acceptable salts include the acid addition salts with the conventional pharmaceutical acids, for example maleic, hydrochloric, hydrobromic, phosphoric, acetic, fumaric, salicylic, citric, lactic, mandelic, tartaric, succinic, benzoic, ascorbic and methanesulphonic.

Suitable pharmaceutically acceptable salts include salts of acidic moieties of the compounds of formula (I) when they are present, for example salts of carboxy groups or phenolic hydroxy groups.

Suitable salts of acidic moieties include metal salts, such as for example aluminium, alkali metal salts such as lithium, sodium or potassium, alkaline earth metal salts such as calcium or magnesium and ammonium or substituted ammonium salts, for example those with lower alkylamines such as triethylamine, hydroxy alkylamines such as 2-hydroxyethylamine, bis-(2-hydroxyethyl)-amine or tri-(2-hydroxyethyl)-amine, cycloalkylamines such as bicyclohexylamine, or with procaine, dibenzylpiperidine, N-benzyl-β-phenethylamine, dehydroabietylamine, N,N′-bisdehydroabietylamine, glucamine, N-methylglucamine or bases of the pyridine type such as pyridine, collidine, quinine or quinoline.

Suitable solvates are pharmaceutically acceptable solvates.

Suitable pharmaceutically acceptable solvates include hydrates.

The term ‘alkyl’ (unless specified to the contrary) when used alone or when forming part of other groups (such as the ‘alkoxy’ group) denotes straight- or branched-chain alkyl groups containing 1 to 12, preferably 1-6 carbon atoms, examples include methyl, ethyl, n-propyl, isopropyl, n-butyl, isobutyl or tert-butyl group.

The term ‘cycloalkyl’ (unless specified to the contrary) when used alone or when forming part of other groups (such as the ‘cycloalkylalkyl’ group) denotes cyclic saturated or unsaturated carbon rings including 3-12, preferably 3-8 carbon ring members. Examples include cyclopropyl, cyclobutyl, cyclohexyl, cyclooctyl.

The term ‘alkenyl’ (unless specified to the contrary) when used alone or when forming part of other groups denotes straight- or branched-unsaturated carbon chains including at least one double C═C bond and containing 2-12, preferably 2-6 carbon atoms.

The term ‘carbocylic’ denotes cycloalkyl and aryl rings.

The term ‘aryl’ denotes aromatic groups including phenyl and naphthyl, preferably phenyl which unless specified to the contrary optionally comprise up to five, preferably up to three substituents selected from halogen, alkyl, phenyl, alkoxy, haloalkyl, hydroxyalkyl, hydroxy, amino, nitro, cyano, carboxy, alkoxycarbonyl, alkoxycarbonylalkyl, alkylcarbonyloxy, or alkylcarbonyl groups.

The term ‘aromatic heterocyclic group’ denotes groups comprising aromatic heterocyclic rings containing from 5 to 12 ring atoms, suitably 5 or 6, and comprising up to four hetero-atoms in the or each ring selected from S, O or N.

Composite terms such as ‘alkylcarboxy’, ‘cycloalkylalkyl’ and so forth refer to components of a compound which include two interlinked groups, with the group named latterly in the term being the linking group, so that ‘alkylcarboxy’ means (alkyl)-COO— whilst ‘cycloalkylalkyl’ means (cycloalkyl)-(alkyl)-.

Unless specified to the contrary, suitable substituents for any heterocyclic group includes up to 4 substituents selected from the group consisting of: alkyl, alkoxy, aryl and halogen or any two substituents on adjacent carbon atoms, together with the carbon atoms to which they are attached, may form an aryl group, preferably a benzene ring, and wherein the carbon atoms of the aryl group represented by the said two substituents may themselves be substituted or unsubstituted.

It will be understood that, unless otherwise specified, groups and substituents forming part of a compound in accordance with the invention are unsubstituted.

When used herein the term “halogen” or “halo” refers to fluorine, chlorine, bromine and iodine, preferably fluorine, chlorine or bromine.

When used herein the term “acyl” includes residues of acids, in particular a residue of a carboxylic acid such as an alkyl- or aryl-carbonyl group.

The invention also provides in one aspect a process for the preparation of a compound of formula (I), or a salt thereof and/or a solvate thereof, which process comprises reacting a compound of formula (II) or an active derivative thereof:

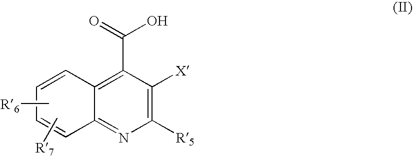

wherein R′₆, R′₇, R′₅ and X′ are R₆, R₇, R₅ and X respectively as hereinbefore defined in relation to formula (I) or (Ia), or a group convertible to R₆, R₇, R₅ and X respectively; with a compound of formula (III): 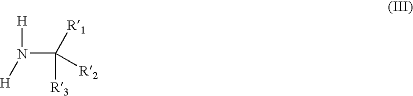
wherein R′₁, R′₂, and R′₃ are R₁, R₂, and R₃ as defined for formula (I) or a group or atom convertible to R₁, R₂, and R₃ respectively; to form a compound of formula (Ib): 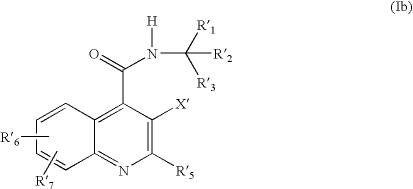
wherein R′₁, R′₂, R′₃, X′, R′₅, R′₆ and R′₇ are as defined above, and thereafter carrying out one or more of the following optional steps:
(i) converting any one of R′₁, R′₂, R′₃, X′, R′₅, R′₆ and R′₇ to R₁, R₂, R₃, X, R₅, R₆ and R₇ respectively as required, to obtain a compound of formula (I);
(ii) converting a compound of formula (I) into another compound of formula (I); and
(iii) preparing a salt of the compound of formula (I) and/or a solvate thereof.

Suitable groups convertible into other groups include protected forms of said groups.

Suitably R′₁, R′₂, R′₃, X′, R′₅, R′₆ and R′₇ each represents R₁, R₂, R₃, X, R₅, R₆ and R₇ respectively or a protected form thereof.

It is favoured if the compound of formula (II) is present as an active derivative.

A suitable active derivative of a compound of formula (II) is a transient activated form of the compound of formula (II) or a derivative wherein the carboxy group of the compound of formula (II) has been replaced by a different group or atom, for example by an acyl halide, preferably a chloride, or an acylazide or a carboxylic acid anhydride.

Other suitable active derivatives include: a mixed anhydride formed between the carboxyl moiety of the compound of formula (II) and an alkyl chloroformate; an activated ester, such as a cyanomethyl ester, thiophenyl ester, p-nitrophenyl ester, p-nitrothiophenyl ester, 2,4,6-trichlorophenyl ester, pentachlorophenyl ester, pentafluorophenyl ester, N-hydroxy-phtalimido ester, N-hydroxypiperidine ester, N-hydroxysuccinimide ester, N-hydroxy benzotriazole ester; alternatively, the carboxy group of the compound of formula (II) may be activated using a carbodiimide or N,N′-carbonyldiimidazole.

The reaction between the compound of formula (II) or the active derivative thereof and the compound of formula (III) is carried out under the appropriate conventional conditions for the particular compounds chosen. Generally, when the compound of formula (II) is present as an active derivative the reaction is carried out using the same solvent and conditions as used to prepare the active derivative, preferably the active derivative is prepared in situ prior to forming the compound of formula (Ib) and thereafter the compound of formula (I) or a salt thereof and/or a solvate thereof is prepared.

For example, the reaction between an active derivative of the compound of formula (II) and the compound of formula (III) may be carried out:

(a) by first preparing an acid chloride and then coupling said chloride with the compound of formula (III) in the presence of an inorganic or organic base in a suitable aprotic solvent such as dimethylformamide (DMF) at a temperature in a range from −70 to 50° C. (preferably in a range from −10 to 20° C.); or

(b) by treating the compound of formula (II) with a compound of formula (III) in the presence of a suitable condensing agent, such as for example N,N′-carbonyl diimidazole (CDI) or a carbodiimide such as dicyclohexylcarbodiimide (DCC) or N-dimethylaminopropyl-N′-ethylcarbodiimide, preferably in the presence of N-hydroxybenzotriazole (HOBT) to maximise yields and avoid racemization processes (see Synthesis, 453, 1972), or O-benzotriazol-1-yl-N,N,N′,N′-tetramethyluroniumhexafluorophosphate (HBTU), in an aprotic solvent, such as a mixture of acetonitrile (MeCN) and tetrahydrofuran (THF), for example a mixture in a volume ratio of from 1:9 to 7:3 (MeCN:THF), at any temperature providing a suitable rate of formation of the required product, such as a temperature in the range of from −70 to 50° C., preferably in a range of from −10 to 25° C., for example at 0° C.

A preferred reaction is set out in Scheme 1 shown below: 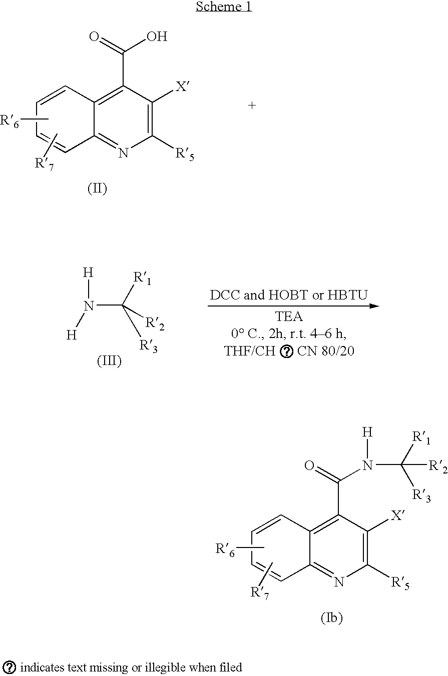
wherein R′₁, R′₂, R′₃, X′, R′₅, R′₆ and R′₇ are as defined above.

In the case in which the corresponding alkyl (such as methyl or ethyl) ester of compound (II) is utilised, an hydrolysis to compound (II) is required before conversion to compound (Ib) in Scheme 1. Such hydrolysis can be carried out under acidic conditions, such 10-36% hydrochloric acid at a temperature in the range between 30 and 100° C.

It will be appreciated that a compound of formula (Ib) may be converted to a compound of formula (I), or one compound of formula (I) may be converted to another compound of formula (I) by interconversion of suitable substituents. Thus, certain compounds of formula (I) and (Ib) are useful intermediates in forming other compounds of the present invention.

Accordingly, in a further aspect the invention provides a process for preparing a compound of formula (I), or a salt thereof and/or a solvate thereof, which process comprises converting a compound of the above defined formula (Ib) wherein at least one of R′₁, R′₂, R′₃, X′, R′₅, R′₆ and R′₇ is not R₁, R₂, R₃, X, R₅, R₆ or R₇ respectively, thereby to provide a compound of formula (I); and thereafter, as required, carrying out one or more of the following optional steps:

(i) converting a compound of formula (I) into another compound of formula (I); and

(ii) preparing a salt of the compound of formula (I) and/or a solvate thereof.

Suitably, in the compound of formula (Ib) the variables R′₁, R′₂, R′₃, X′, R′₅, R′₆ and R′₇ are R₁, R₂, R₃, X, R₅, R₆ and R₇ respectively or they are protected forms thereof.

The above mentioned conversions, protections and deprotections are carried out using the appropriate conventional reagents and conditions and are further discussed below.

A chiral compound of formula (III) wherein R₂ is a C₅ or C₇ cycloalkyl group, R₃ is methyl and R₁ is H are described in J. Org. Chem. (1996), 61 (12), 4130-4135. A chiral compound of formula (III) wherein R₂ is phenyl, R₃ is isopropyl and R₁ is H is a known compound described in for example Tetrahedron Lett. (1994), 35(22), 3745-6.

The compounds of formula (III) are known commercially available compounds or they can be prepared from known compounds by known methods, or methods analogous to those used to prepare known compounds, for example the methods described in Liebigs Ann. der Chemie, (1936), 523, 199.

In some embodiments of the invention, a compound of formula (II) or the corresponding alkyl (such as methyl or ethyl) ester is prepared by reacting a compound of formula (IV) or the corresponding alkyl (such as methyl or ethyl) ester: 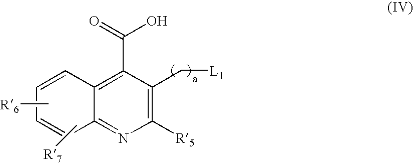
wherein R′₆, R′₇, R′₅ and a are as defined above and L₁ represents a halogen atom such as a bromine atom, with a compound of formula (V): 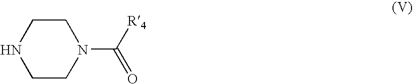
wherein R′₄ is R₄ as defined in relation to formula (I) or a protected form thereof.

Suitably, R′₄ is R₄.

Suitably, reaction between the compounds of formulae (IV) or the corresponding alkyl (such as methyl or ethyl) ester and (V) is carried out under conventional amination conditions, for example when L₁ is a bromine atom then the reaction is conveniently carried out in an aprotic solvent, such as tetrahydrofuran or dimethylformamide at any temperature providing a suitable rate of formation of the required product, usually at ambient temperature; preferably the reaction is carried out in the presence of triethylamine (TEA) or K₂CO₃.

The compounds of formula (V) are known, commercially available compounds or they can be prepared using methods analogous to those used to prepare known compounds; for example the methods described in the Chemistry of the Amino Group, Patais (Ed.), Interscience, New York 1968; Advanced Organic Chemistry, March J, John Wiley & Sons, New York, 1992; J. Heterocyclic Chem. (1990), 27, 1559; Synthesis (1975), 135, Bioorg. Med. Chem. Lett. (1997), 7, 555, or Protective Groups in Organic Synthesis (second edition), Wiley Interscience, (1991) or other methods mentioned herein.

In cases where a is 1, a compound of formula (IV) or the corresponding alkyl (such as methyl or ethyl) ester may be prepared by appropriate halogenation of a compound of formula (VI) or the corresponding alkyl (such as methyl or ethyl) ester: 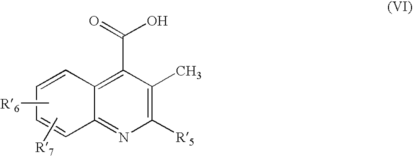
wherein R′₆, R′₇ and R′₅ are as defined above in relation to formula (II).

Suitable halogenation reagents are conventional reagents depending upon the nature of the halogen atom required, for example when L₁ is bromine a preferred halogenation reagent is N-bromosuccinimide (NBS).

The halogenation of the compound of formula (VI) or the corresponding alkyl (such as methyl or ethyl) ester is suitably carried out under conventional conditions, for example bromination is carried out by treatment with NBS in an inert solvent, such as carbon tetrachloride CCl₄, or 1,2-dichloroethane or CH₃CN, at any temperature providing a suitable rate of formation of the required product, suitably at an elevated temperature such as a temperature in the range of 60° C. to 100° C., for example 80° C.; preferably the reaction is carried out in the presence of a catalytic amount of benzoyl peroxide.

A compound of formula (VI) is conveniently prepared by reacting a compound of formula (VII): 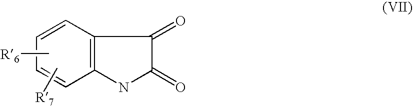
wherein R′₆ and R′₇ are as defined in relation to formula (II), with a compound of formula (XIII):
R₅′—CO—CH₂—Me (XIII)
wherein R′₅ is as defined in relation to formula (II).

The reaction between the compounds of formula (VII) and (XIII) is conveniently carried out using Pfitzinger reaction conditions (see for example J. Prakt. Chem. 33, 100 (1886), J. Prakt. Chem. 38, 582 (1888), J. Chem. Soc. 106 (1948) and Chem. Rev. 35, 152 (1944)), for example in an alkanolic solvent such as ethanol, at any temperature providing a suitable rate of formation of the required product, but generally at an elevated temperature, such as the reflux temperature of the solvent, and preferably in the presence of a base such as potassium hydroxide or potassium tert-butoxide.

The compounds of formula (VII) are known compounds or they are prepared according to methods used to prepare known compounds for example those disclosed in J. Org. Chem. 21, 171 (1955); J. Org. Chem. 21, 169 (1955).

Alternatively a compound of formula (VI) may be conveniently prepared by reacting a compound of formula (XIV) 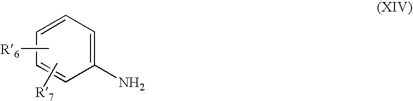
wherein R′₆ and R′₇ are as defined in relation to formula (II), with a compound of formula (XV):
R₅′—CHO (XV)
wherein R′₅ is as defined in relation to formula (II) in presence of oxobutyric acid.

The reaction between the compounds of formula (XIV) and (XV) is conveniently carried out using Doebner reaction conditions (see for example Chem. Ber. 29, 352 (1894); Chem. Revs. 35, 153, (1944); J. Chem. Soc. B, 1969, 805), for example in an alcoholic solvent such as ethanol, at any temperature providing a suitable rate of formation of the required product, but generally at an elevated temperature, such as the reflux temperature of the solvent.

The compounds of formula (XIV) and (XV) are known compounds or they are prepared according to methods used to prepare known compounds for example as described in Vogel's Textbook of Practical Organic Chemistry.

In some alternative embodiments of the invention, a compound of formula (II) wherein X′ represents 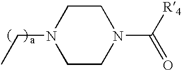
is prepared by reacting a compound of formula (VII) as defined above with a compound of formula (VIII):
R₅′—CO—CH₂—(CH₂)a-T₅ (VIII)
wherein R′₅ is as defined in relation to formula (II), and T₅ is a group 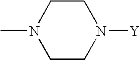
where Y is a protecting group such as a benzyl group, particularly a protecting group which is stable in basic conditions such as a terbutoxycarbonyl group, or a group COR₄ as defined in relation to formula (I) or a protected form thereof or a group convertible thereto, and a is as defined in relation to formula (II); and thereafter as required removing any protecting group, for example by dehydrogenation, and/or converting any group T₅ to

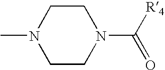

The reaction between the compounds of formula (VII) and (VIII) is conveniently carried out using Pfitzinger reaction conditions (see for example J. Prakt. Chem. 33, 100 (1886), J. Prakt. Chem. 38, 582 (1888), J. Chem. Soc. 106 (1948) and Chem. Rev. 35, 152 (1944)), for example in an alkanolic solvent such as ethanol, at any temperature providing a suitable rate of formation of the required product, but generally at an elevated temperature, such as the reflux temperature of the solvent, and preferably in the presence of a base such as potassium hydroxide or potassium tert-butoxide.

Protected forms of 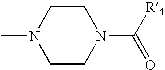
will vary according to the particular nature of the group being protected but will be chosen in accordance with normal chemical practice.

Groups convertible to

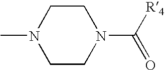

include groups dictated by conventional chemical practice to be required and to be appropriate, depending upon the specific nature of the

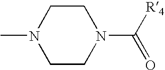

under consideration.
Suitable deprotection methods for deprotecting protected forms of 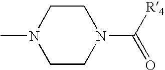
and conversion methods for converting T₅ to 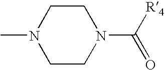
will be those used conventionally in the art depending upon the particular groups under consideration with reference to standard texts such as Greene, T. W. and Wuts, P. G. M. Protective Groups in Organic Synthesis, John Wiley & Sons Inc. New York, 1991 (Second Edt.) or in Kocienski, P. J. Protecting groups. George Thieme Verlag, New York, 1994 and Chemistry of the Amino Group, Patais (Ed.), Interscience, New York 1968; or Advanced Organic Chemistry, March J, John Wiley & Sons, New York, 1992.

A compound of formula (VIII) is prepared from a compound of formula (IX):
R₅′—CO—CH₂—(CH₂)ₐ—OH (IX)
wherein R′₅ is as defined in relation to formula (II) and a is as defined in relation to formula (VIII), by first halogenating, preferably brominating, or mesylating the compound of formula (IX) and thereafter reacting the halogenation or mesylation product so formed with a compound capable of forming a group T₅ so as to provide the required compound of formula (VII).

When T₅ is a group 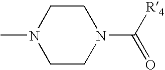
a compound capable of forming a group T₅ is a compound of the above defined formula (V).

The halogenation of the compound of formula (IX) is suitably carried out using a conventional halogenation reagent. Mesylation is conveniently carried out using mesyl chloride in an inert solvent such as methylene dichloride, at a temperature below room temperature, such as 0° C., preferably in the presence of triethylamine.

The reaction conditions between the compound of formula (IX) and the compound capable of forming a group T₅ will be those conventional conditions dictated by the specific nature of the reactants, for example when the T₅ required is a group 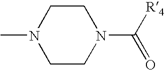
and the required compound capable of forming a group T₅ is a compound of the above defined formula (V), then the reaction between the halogenation or mesylation product of the compound of formula (IX) and the compound of formula (V) is carried out under analogous conditions to those described for the reaction between the compounds of formulae (IV) and (V).

Other compounds capable of forming a group T₅ will depend upon the particular nature of T₅, but will be those appropriate compounds dictated by conventional chemical practice with reference to standard texts such as Chemistry of the Amino Group, Patais (Ed.), Interscience, New York 1968; and Advanced Organic Chemistry, March J, John Wiley & Sons, New York, 1992.

A compound of formula (IX) may be prepared by reacting a compound of formula (X): 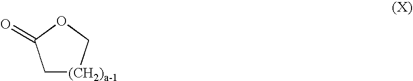
wherein a is as defined in relation to formula (VIII), with a lithium salt of formula (XI):
R′₅Li (XI)
wherein R′₅ is as defined in relation to formula (II).

The reaction between the compounds of formulae (X) and (XI) can be carried out in an aprotic solvent, such as diethyl-ether at any temperature providing a suitable rate of formation of the required product, usually at a low temperature such as in the range of −10° C. to −30° C., for example −20° C.

The compounds of formula (VII) are known compounds or they are prepared according to methods used to prepare known compounds for example those disclosed in J. Org. Chem. 21, 171 (1955); J. Org. Chem. 21, 169 (1955).

The compounds of formula (X) and (XI) are known compounds or they are prepared according to methods used to prepare known compounds for example those disclosed by Krow G. R. in Organic Reactions, Vol 43, page 251, John Wiley & Sons Inc. 1994 (for the compounds of formula (X)) and Organometallics in Synthesis, Schlosser M.(Ed), John Wiley & Sons Inc. 1994 (for the compounds of formula (XI)).

In another aspect, the present invention provides a process for the preparation of a compound of formula (I), or a salt thereof and/or a solvate thereof, wherein a is 1, which process comprises reacting a compound of formula (XVI): 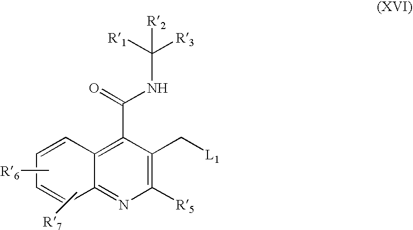
wherein each of R′₁, R′₂, R′₃, R′₅, R′₆, and R′₇ is respectively R₁, R₂, R₃, R₅, R₆, or R₇ as defined above or a group convertible to R₁, R₂, R₃, R₅, R₆, or R₇ respectively as defined above providing R′₂ is not aromatic in character, and L₁ represents a halogen atom such as a bromine atom, with a compound of formula (XVII): 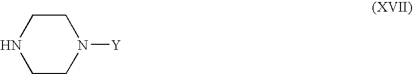
wherein Y is a protecting group such as a benzyl group, particularly a protecting group which is stable in basic conditions such as a terbutoxycarbonyl group, or a group COR′₄, where R′₄ is R₄ as defined in relation to formula (I) or a protected form thereof or a group convertible thereto; and thereafter as required removing any protecting group Y, for example by dehydrogenation, and replacing the protective group Y with a group COR′₄; and thereafter carrying out one or more of the following optional steps:
(i) converting any one of R′₁, R′₂, R′₃, R′₄, R′₅, R′₆ and R′₇ to R₁, R₂, R₃, R₄, R₅, R₆ and R₇ respectively as required, to obtain a compound of formula (I);
(ii) converting a compound of formula (I) into another compound of formula (I); and
(iii) preparing a salt of the compound of formula (I) and/or a solvate thereof.

Protected forms of R₄ will vary according to the particular nature of the group being protected but will be chosen in accordance with normal chemical practice.

Groups convertible to R₄ include groups dictated by conventional chemical practice to be required and to be appropriate, depending upon the specific nature of the R₄ under consideration.

Suitable deprotection methods for deprotecting protected forms of R₄ and conversion methods for converting R′₄ to R₄ will be those used conventionally in the art depending upon the particular groups under consideration with reference to standard texts such as Greene, T. W. and Wuts, P. G. M. Protective Groups in Organic Synthesis, John Wiley & Sons Inc. New York, 1991 (Second Edt.) or in Kocienski, P. J. Protecting groups. George Thieme Verlag, New York, 1994 and Chemistry of the Amino Group, Patais (Ed.), Interscience, New York 1968; or Advanced Organic Chemistry, March J, John Wiley & Sons, New York, 1992.

Suitable groups convertible into other groups include protected forms of said groups.

Advantageously, a compound of formula (XVII) will be a compound of formula (V) as defined above.

Suitably R′₁, R′₂, R′₃, R′₄, R′₅, R′₆ and R′₇ each represents R₁, R₂, R₃, R₄, R₅, R₆ and R₇ respectively or a protected form thereof.

Suitable deprotection methods for deprotecting protected forms of R₁, R₂, R₃, R₄, R₅, R₆ and R₇ and conversion methods for converting R′₁, R′₂, R′₃, R′₄, R′₅, R′₆ and R′₇ to R₁, R₂, R₃, R₄, R₅, R₆ and R₇ respectively will be those used conventionally in the art depending upon the particular groups under consideration with reference to standard texts such as Greene, T. W. and Wuts, P. G. M. Protective Groups in Organic Synthesis, John Wiley & Sons Inc. New York, 1991 (Second Edt.) or in Kocienski, P. J. Protecting groups. George Thieme Verlag, New York, 1994 and Chemistry of the Amino Group, Patais (Ed.), Interscience, New York 1968; or Advanced Organic Chemistry, March J, John Wiley & Sons, New York, 1992.

Suitably, reaction between the compounds of formulae (XVI) and (XVII) is carried out under conventional amination conditions, for example when L₁ is a bromine atom then the reaction is conveniently carried out in an aprotic solvent, such as tetrahydrofuran or dimethylformamide at any temperature providing a suitable rate of formation of the required product, usually at ambient temperature; preferably the reaction is carried out in the presence of triethylamine (TEA) or K₂CO₃.

The compounds of formula (XVII) are known, commercially available compounds or they can be prepared using methods analogous to those used to prepare known compounds; for example the methods described in the Chemistry of the Amino Group, Patais (Ed.), Interscience, New York 1968; Advanced Organic Chemistry, March J, John Wiley & Sons, New York, 1992; J. Heterocyclic Chem. (1990), 27, 1559; Synthesis (1975), 135, Bioorg. Med. Chem. Lett. (1997), 7, 555, or Protective Groups in Organic Synthesis (second edition), Wiley Interscience, (1991) or other methods mentioned herein.

A compound of formula (XVI) is prepared by appropriate halogenation of a compound of formula (XVIII): 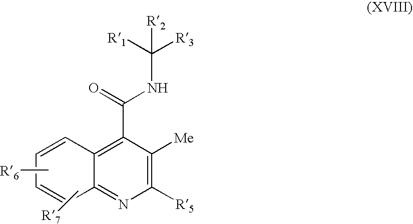
wherein R′₁, R′₂, R′₃, R′₅, R′₆, and R′₇ are as defined above in relation to formula (XVI).

Suitable halogenation reagents are conventional reagents depending upon the nature of the halogen atom required, for example when L₁ is bromine a preferred halogenation reagent is N-bromosuccinimide (NBS).

The halogenation of the compound of formula (XVIII) is carried out under conventional conditions, for example bromination is carried out by treatment with NBS in an inert solvent, such as carbon tetrachloride CCl₄, or 1,2-dichloroethane or CH₃CN, at any temperature providing a suitable rate of formation of the required product, suitably at an elevated temperature such as a temperature in the range of 60° C. to 100° C., for example 80° C.; preferably the reaction is carried out in the presence of a catalytic amount of benzoyl peroxide.

Suitably, the compound of formula (XVIII) may be prepared by reacting a compound of formula (VI) as defined above or an active derivative thereof with a compound of formula (III) as defined above wherein R′₂ is not aromatic in character.

It is favoured if the compound of formula (VI) is present in the reaction mix as an active derivative, as hereinbefore described.

The reaction between the compound of formula (VI) or the active derivative thereof and the compound of formula (III) is carried out under the appropriate conventional conditions for the particular compounds chosen. Generally, when the compound of formula (VI) is present as an active derivative the reaction is carried out using the same solvent and conditions as used to prepare the active derivative, preferably the active derivative is prepared in situ prior to forming the compound of formula (XVIII).

For example, the reaction between an active derivative of the compound of formula (VI) and the compound of formula (III) may be carried out:

(a) by first preparing an acid chloride and then coupling said chloride with the compound of formula (III) in the presence of an inorganic or organic base in a suitable aprotic solvent such as dimethylformamide (DMF) at a temperature in a range from −70 to 50° C. (preferably in a range from −10 to 20° C.); or

(b) by treating the compound of formula (VI) with a compound of formula (III) in the presence of a suitable condensing agent, such as for example N,N′-carbonyl diimidazole (CDI) or a carbodiimide such as dicyclohexylcarbodiimide (DCC) or N-dimethylaminopropyl-N′-ethylcarbodiimide, preferably in the presence of N-hydroxybenzotriazole (HOBT) to maximise yields and avoid racemization processes (see Synthesis, 453, 1972), or O-benzotriazol-1-yl-N,N,N′,N′-tetramethyluroniumhexafluorophosphate (HBTU), in an aprotic solvent, such as a mixture of acetonitrile (MeCN) and tetrahydrofuran (THF), for example a mixture in a volume ratio of from 1:9 to 7:3 (MeCN:THF), at any temperature providing a suitable rate of formation of the required product, such as a temperature in the range of from −70 to 50° C., preferably in a range of from −10 to 25° C., for example at 0° C.

A preferred reaction is set out in Scheme 2 shown below:

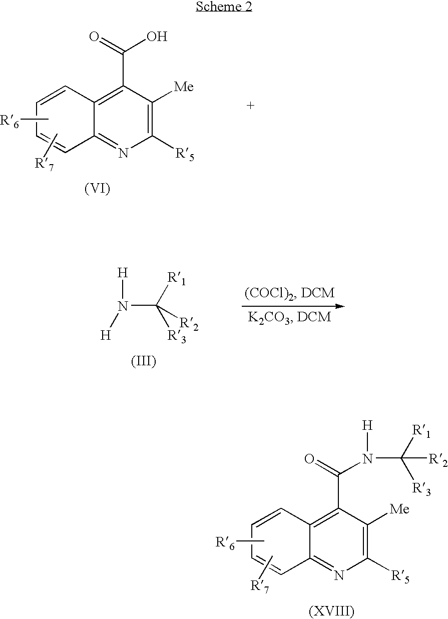

In the case in which the corresponding alkyl (such as methyl or ethyl) ester of compounds (VI) is utilised, a hydrolysis is required before conversion to compound (XVIII) in Scheme 2. Such hydrolysis can be carried out under acidic conditions, such 10-36% hydrochloric acid at a temperature in the range between 30 and 100° C.

In yet further embodiments, compounds of formula (Ib) can be prepared by reacting a compound of formula XIX 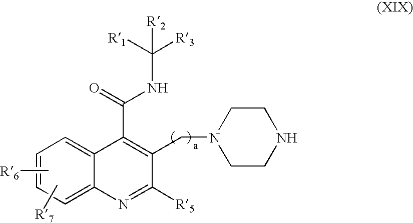
wherein R′₁, R′₂, R′₃, R′₅, R′₆, R′₇ and a are as defined above, with a compound of formula (XX)

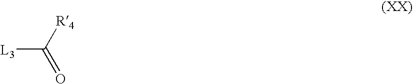

wherein L₃ represents a leaving group for example halogen or activated ester, preferably chlorine, bromine or p-nitrophenylester and R′₄ represents R₄ as defined in relation to formula (I) or a protected form thereof or a group convertible thereto.

Ureas or substituted ureas of formula I are best prepared by reacting compounds of formula (XIX) with metal cyanates such as potassium or sodium cyanate or with substituted isocyanates, following scheme 3 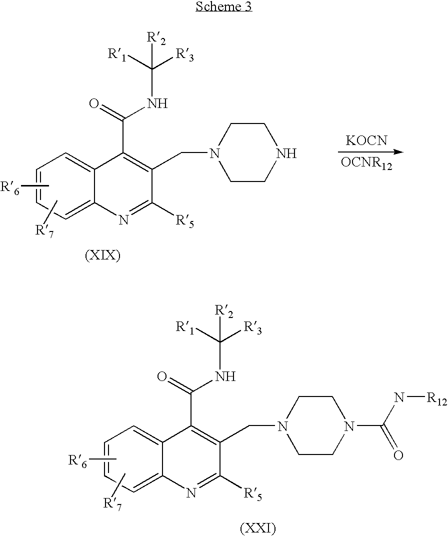
wherein R₁₂ represents H, lower alkyl, optionally substituted aryl or aralkyl.

Compounds of formula (XIX) are prepared by removing the protective group of a compound of formula (XXII) 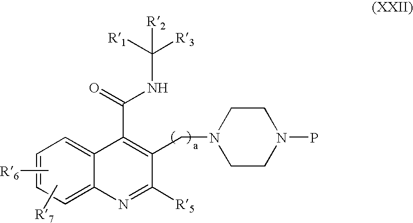
wherein R′₁, R′₂, R′₃, R′₅, R′₆, R′₇, and a are as defined above and P is an amine protective group, for example fmoc or benzyl, preferably fmoc. The protective group is removed by standard methods described in the literature, for example the fmoc residue is splitted by action of piperidine at room temperature in a solvent like acetonitrile.

As hereinbefore mentioned, the compounds of formula (I) may exist in more than one stereoisomeric form—and the process of the invention may produce racemates as well as enantiomerically pure forms. Accordingly, a pure enantiomer of a compound of formula (I) can be obtained by reacting a compound of the above defined formula (II) with an appropriate enantiomerically pure primary amine of formula (IIIa) or (IIIc):

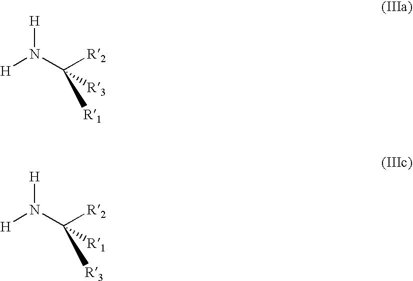

wherein R′₁, R′₂ and R′₃ are as defined above, to obtain a compound of formula (I′a) or (I′c):

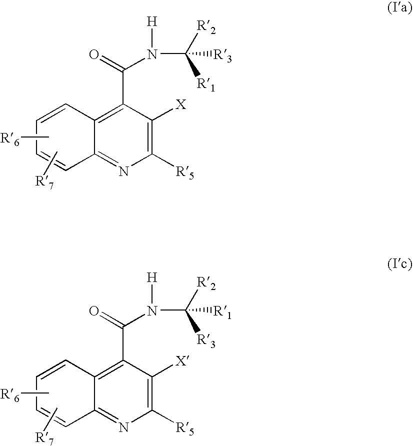

wherein R′₁, R′₂, R′₃, X′, R′₅, R′₆, and R′₇ are as defined above.

Compounds of formula (I′a) or (I′c) may subsequently be converted to compounds of formula (Ia) or (Ic) by the methods of conversion mentioned before:

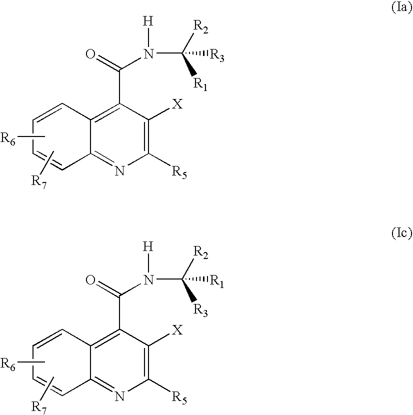

wherein R₁, R₂, R₃, X, R₅, R₆, and R₇ are as defined above.

Suitably, in the above mentioned compounds of formulae (Ia), (Ic), (I′a), (I′c), (IIIa) and (IIIc) R₁ represents hydrogen.

An alternative method for separating optical isomers is to use conventional, fractional separation methods in particular fractional crystallization methods. Thus, a pure enantiomer of a compound of formula (I) is obtained by fractional crystallisation of a diastereomeric salt formed by reaction of the racemic compound of formula (I) with an optically active strong acid resolving agent, such as camphosulphonic acid, tartaric acid, O,O′-di-p-toluoyltartaric acid or mandelic acid, in an appropriate alcoholic solvent, such as ethanol or methanol, or in a ketonic solvent, such as acetone. The salt formation process should be conducted at a temperature between 20° C. and 80° C., preferably at 50° C.

A suitable conversion of one compound of formula (I) into a further compound of formula (I) involves converting one group X into another group X by for example:

(i) converting a ketal into a ketone, by such as mild acidic hydrolysis, using for example dilute hydrochloric acid;

(ii) reducing a ketone to a hydroxy group by use of a borohydride reducing agent;

(iii) converting a carboxylic ester group into a carboxyl group using basic hydrolysis; and/or

(iv) reducing a carboxylic ester group to a hydroxymethyl group, by use of a borohydride reducing agent.

As indicated above, where necessary, the conversion of any group R′₁, R′₂, R′₃, X′, R′₅, R′₆, and R′₇ into R₁, R₂, R₃, X, R₅, R₆, and R₇ which as stated above are usually protected forms of R₁, R₂, R₃, X, R₅, R₆, or R₇ may be carried out using appropriate conventional conditions such as the appropriate deprotection procedure.

It will be appreciated that in any of the above mentioned reactions any reactive group in the substrate molecule may be protected and deprotected according to conventional chemical practice, for example as described by Greene, T. W. and Wuts, P. G. M. Protective Groups in Organic Synthesis, John Wiley & Sons Inc. New York, 1991 (Second Edt.) or in Kocienski, P. J. Protecting groups. George Thieme Verlag, New York, 1994.

Suitable protecting groups in any of the above mentioned reactions are those used conventionally in the art. Thus, for example suitable hydroxy protecting groups include benzyl or trialkylsilyl groups.

The methods of formation and removal of such protecting groups are those conventional methods appropriate to the molecule being protected. Thus for example a benzyloxy group may be prepared by treatment of the appropriate compound with a benzyl halide, such as benzyl bromide, and thereafter, if required, the benzyl group may be conveniently removed using catalytic hydrogenation or a mild ether cleavage reagent such as trimethylsilyl iodide or boron tribromide.

As indicated above, the compounds of formula (I) have useful pharmaceutical properties.

Accordingly the present invention also provides a compound of formula (I), or a pharmaceutically acceptable salt or solvate thereof, for use as an active therapeutic substance.

In particular, the present invention also provides a compound of formula (I), or a pharmaceutically acceptable salt or solvate thereof, for the treatment or prophylaxis of the Primary and Secondary Conditions.

The present invention further provides a pharmaceutical composition comprising a compound of formula (I), or a pharmaceutically acceptable salt or solvate thereof, and a pharmaceutically acceptable carrier.

The present invention also provides the use of a compound of formula (I), or a pharmaceutically acceptable salt or solvate thereof, in the manufacture of a medicament for the treatment of the Primary and Secondary Conditions.

As mentioned above the Primary conditions include respiratory diseases, such as chronic obstructive pulmonary disease (COPD), asthma, airway hyper-reactivity, cough; inflammatory diseases such as inflammatory bowel disease, psoriasis, fibrositis, osteoarthritis, rheumatoid arthritis and inflammatory pain; neurogenic inflammation or peripheral neuropathy, allergies such as eczema and rhinitis; ophthalmic diseases such as ocular inflammation, conjunctivitis, vernal conjuctivitis and the like; cutaneous diseases, skin disorders and itch, such as cutaneous wheal and flare, contact dermatitis, atopic dermatitis, urticaria and other eczematoid dermatitis; adverse immunological reactions such as rejection of transplanted tissues and disorders related to immune enhancement or suppression such as systhemic lupus erythematosis; gastrointestinal (GI) disorders and diseases of the GI tract such as disorders associated with the neuronal control of viscera such as ulcerative colitis, Crohn's disease, irritable bowel syndrome (IBS), gastro-exophageous reflex disease (GERD); urinary incontinence and disorders of the bladder function; renal disorders; increased blood pressure, proteinuria, coagulopathy and peripheral and cerebral oedema following pre-eclampsia in pregnancies.

As mentioned above, the Secondary conditions include disorders of the central nervous system such as anxiety, depression, psychosis and schizophrenia; neurodegenerative disorders such as AIDS related dementia, senile dementia of the Alzheimer type, Alzheimer's disease, Down's syndrome, Huntingdon's disease, Parkinson's disease, movement disorders and convulsive disorders (for example epilepsy); demyelinating diseases such as multiple sclerosis and amyotrophic lateral sclerosis and other neuropathological disorders such as diabetic neuropathy, AIDS related neuropathy, chemotherapy-induced neuropathy and neuralgia; addiction disorders such as alcoholism; stress related somatic disorders; reflex sympathetic dystrophy such as shoulder/hand syndrome; dysthymic disorders; eating disorders (such as food intake disease); fibrosing and collagen diseases such as scleroderma and eosinophilic fascioliasis; disorders of the blood flow caused by vasodilatation and vasospastic diseases such as angina, migraine and Reynaud's disease and pain or nociception, for example, that is attributable to or associated with any of the foregoing conditions especially the transmission of pain in migraine.

Such a medicament, and a composition of this invention, may be prepared by admixture of a compound of the invention with an appropriate carrier. It may contain a diluent, binder, filler, disintegrant, flavouring agent, colouring agent, lubricant or preservative in conventional manner.

These conventional excipients may be employed for example as in the preparation of compositions of known agents for treating the conditions.

Preferably, a pharmaceutical composition of the invention is in unit dosage form and in a form adapted for use in the medical or veterinarial fields. For example, such preparations may be in a pack form accompanied by written or printed instructions for use as an agent in the treatment of the conditions.

The suitable dosage range for the compounds of the invention depends on the compound to be employed and on the condition of the patient. It will also depend, inter alia, upon the relation of potency to absorbability and the frequency and route of administration.

The compound or composition of the invention may be formulated for administration by any route, and is preferably in unit dosage form or in a form that a human patient may administer to himself in a single dosage. Advantageously, the composition is suitable for oral, rectal, topical, parenteral, intravenous or intramuscular administration. Preparations may be designed to give slow release of the active ingredient.

Compositions may, for example, be in the form of tablets, capsules, sachets, vials, powders, granules, lozenges, reconstitutable powders, or liquid preparations, for example solutions or suspensions, or suppositories.

The compositions, for example those suitable for oral administration, may contain conventional excipients such as binding agents, for example syrup, acacia, gelatine, sorbitol, tragacanth, or polyvinylpyrrolidone; fillers, for example lactose, sugar, maize-starch, calcium phosphate, sorbitol or glycine; tabletting lubricants, for example magnesium stearate; disintegrants, for example starch, polyvinyl-pyrrolidone, sodium starch glycollate or microcrystalline cellulose; or pharmaceutically acceptable setting agents such as sodium lauryl sulphate.

Solid compositions may be obtained by conventional methods of blending, filling, tabletting or the like. Repeated blending operations may be used to distribute the active agent throughout those compositions employing large quantities of fillers. When the composition is in the form of a tablet, powder, or lozenge, any carrier suitable for formulating solid pharmaceutical compositions may be used, examples being magnesium stearate, starch, glucose, lactose, sucrose, rice flour and chalk. Tablets may be coated according to methods well known in normal pharmaceutical practice, in particular with an enteric coating. The composition may also be in the form of an ingestible capsule, for example of gelatine containing the compound, if desired with a carrier or other excipients.

Compositions for oral administration as liquids may be in the form of, for example, emulsions, syrups, or elixirs, or may be presented as a dry product for reconstitution with water or other suitable vehicle before use. Such liquid compositions may contain conventional additives such as suspending agents, for example sorbitol, syrup, methyl cellulose, gelatine, hydroxyethylcellulose, carboxymethylcellulose, aluminium stearate gel, hydrogenated edible fats; emulsifying agents, for example lecithin, sorbitan monooleate, or acacia; aqueous or non-aqueous vehicles, which include edible oils, for example almond oil, fractionated coconut oil, oily esters, for example esters of glycerine, or propylene glycol, or ethyl alcohol, glycerine, water or normal saline; preservatives, for example methyl or propyl p-hydroxybenzoate or sorbic acid; and if desired conventional flavouring or colouring agents.

The compounds of this invention may also be administered by a non-oral route. In accordance with routine pharmaceutical procedure, the compositions may be formulated, for example for rectal administration as a suppository. They may also be formulated for presentation in an injectable form in an aqueous or non-aqueous solution, suspension or emulsion in a pharmaceutically acceptable liquid, e.g. sterile pyrogen-free water or a parenterally acceptable oil or a mixture of liquids. The liquid may contain bacteriostatic agents, anti-oxidants or other preservatives, buffers or solutes to render the solution isotonic with the blood, thickening agents, suspending agents or other pharmaceutically acceptable additives. Such forms will be presented in unit dose form such as ampoules or disposable injection devices or in multi-dose forms such as a bottle from which the appropriate dose may be withdrawn or a solid form or concentrate which can be used to prepare an injectable formulation.

The compounds of this invention may also be administered by inhalation, via the nasal or oral routes. Such administration can be carried out with a spray formulation comprising a compound of the invention and a suitable carrier, optionally suspended in, for example, a hydrocarbon propellant.

Preferred spray formulations comprise micronised compound particles in combination with a surfactant, solvent or a dispersing agent to prevent the sedimentation of suspended particles. Preferably, the compound particle size is from about 2 to 10 microns.

A further mode of administration of the compounds of the invention comprises transdermal delivery utilising a skin-patch formulation. A preferred formulation comprises a compound of the invention dispersed in a pressure sensitive adhesive which adheres to the skin, thereby permitting the compound to diffuse from the adhesive through the skin for delivery to the patient. For a constant rate of percutaneous absorption, pressure sensitive adhesives known in the art such as natural rubber or silicone can be used.

As mentioned above, the effective dose of compound depends on the particular compound employed, the condition of the patient and on the frequency and route of administration. A unit dose will generally contain from 20 to 1000 mg and preferably will contain from 30 to 500 mg, in particular 50, 100, 150, 200, 250, 300, 350, 400, 450, or 500 mg. The composition may be administered once or more times a day for example 2, 3 or 4 times daily, and the total daily dose for a 70 kg adult will normally be in the range 100 to 3000 mg. Alternatively the unit dose will contain from 2 to 20 mg of active ingredient and be administered in multiples, if desired, to give the preceding daily dose.

No unacceptable toxicological effects are expected with compounds of the invention when administered in accordance with the invention.

The present invention also provides a method for the treatment and/or prophylaxis of the Primary and Secondary Conditions in mammals, particularly humans, which comprises administering to the mammal in need of such treatment and/or prophylaxis an effective, non-toxic pharmaceutically acceptable amount of a compound of formula (I) or a pharmaceutically acceptable salt or solvate thereof.

The activity of the compounds of the present invention, as NK₃ ligands, is determined by their ability to inhibit the binding of the radiolabelled NK₃ ligands, [¹²⁵I]-[Me-Phe⁷]-NKB or [³H]-Senktide, to guinea-pig and human NK₃ receptors (Renzetti et al, 1991, Neuropeptide, 18, 104-114; Buell et al, 1992, FEBS, 299(1), 90-95; Chung et al, 1994, Biochem. Biophys. Res. Commun., 198(3), 967-972).

The binding assays utilized allow the determination of the concentration of the individual compound required to reduce by 50% the [¹²⁵I]-[Me-Phe⁷]-NKB and [³H]-Senktide specific binding to NK₃ receptor in equilibrium conditions (IC50).

Binding assays provide for each compound tested a mean IC₅₀ value of 2-5 separate experiments performed in duplicate or triplicate. The most potent compounds of the present invention show IC₅₀ values in the range 0.1-1000 nM. The NK₃-antagonist activity of the compounds of the present invention is determined by their ability to inhibit senktide-induced contraction of the guinea-pig ileum (Maggi et al, 1990, Br. J. Pharmacol., 101, 996-1000) and rabbit isolated iris sphincter muscle (Hall et al., 1991, Eur. J. Pharmacol., 199, 9-14) and human NK₃ receptors-mediated Ca⁺⁺ mobilisation (Mochizuki et al, 1994, J. Biol. Chem., 269, 9651-9658). Guinea-pig and rabbit in-vitro functional assays provide for each compound tested a mean K_{B} value of 3-8 separate experiments, where K_{B} is the concentration of the individual compound required to produce a 2-fold rightward shift in the concentration-response curve of senktide. Human receptor functional assay allows the determination of the concentration of the individual compound required to reduce by 50% (IC₅₀ values) the Ca⁺⁺ mobilisation induced by the agonist NKB. In this assay, the compounds of the present invention behave as antagonists.

The activity of the compounds of the present invention, as NK-2 ligands, is determined by their ability to inhibit the binding of the radiolabelled NK-2 ligands, [¹²⁵I]-NKA or [³H]-NKA, to human NK-2 receptors (Aharony et al, 1992, Neuropeptide, 23, 121-130).

The binding assays utilised allow the determination of the concentration of the individual compound required to reduce by 50% the [¹²⁵I]-NKA and [³H]-NKA specific binding to NK2 receptor in equilibrium conditions (IC₅₀).

Binding assays provide for each compound tested a mean IC₅₀ value of 2-5 separate experiments performed in duplicate or triplicate. The most potent compounds of the present invention show IC₅₀ values in the range 0.5-1000 nM, such as 1-1000 nM. The NK-2-antagonist activity of the compounds of the present invention is determined by their ability to inhibit human NK-2 receptor-mediated Ca⁺⁺ mobilisation (Mochizuki et al, 1994, J. Biol. Chem., 269, 9651-9658). Human receptor functional assay allows the determination of the concentration of the individual compound required to reduce by 50% (IC₅₀ values) the Ca⁺⁺ mobilisation induced by the agonist NKA. In this assay, the compounds of the present invention behave as antagonists.

The therapeutic potential of the compounds of the present invention in treating the conditions can be assessed using rodent disease models.

As stated above, the compounds of formula (I) are also considered to be useful as diagnostic tool. Accordingly, the invention includes a compound of formula (I) for use as diagnostic tools for assessing the degree to which neurokinin-3 and neurokinin-2 receptor activity (normal, overactivity or underactivity) is implicated in a patient's symptoms. Such use comprises the use of a compound of formula (I) as an antagonist of said activity, for example including but not restricted to Tachykinin agonist-induced inositol phosphate turnover or electrophysiological activation, of a cell sample obtained from a patient. Comparison of such activity in the presence or absence of a compound of formula (I), will disclose the degree of NK-3 and NK-2 receptor involvement in the mediation of agonist effects in that tissue.

The following Descriptions illustrate the preparation of the intermediates, whereas the following Examples illustrate the preparation of the compounds of the invention.

### DESCRIPTIONS AND EXAMPLES

### Description A

### 3-Methyl-2-phenyl-quinoline-4-carboxylic acid methyl ester

30 g (114 mmol) of 3-methyl-2-phenyl-quinoline-4-carboxylic acid (CAS [43071-45-0]) were suspended in 250 ml of dry CH₂Cl₂; 20 ml (230 mmol) of oxalyl chloride dissolved in 120 ml of CH₂Cl₂ were added dropwise and the reaction mixture was stirred at room temperature for 30 min. Two drops of N,N-dimethylformamide (DMF) were added and the reaction was stirred for additional 30 min. The solvent was evaporated in vacuo to dryness, the residue was taken up with 100 ml of CH₂Cl₂ and 100 ml of MeOH, dissolved in 400 ml of CH₂Cl₂, were added dropwise. After stirring for 18 h, the solvent was evaporated in vacuo to dryness, the residue was taken up with CH₂Cl₂ and washed with 1% NaHCO₃; the organic layer was dried over Na₂SO₄, filtered and evaporated in vacuo to dryness to yield 31.6 g of the title compound as a solid, which was used in the following reaction without further purification.

C₁₈H₁₅NO₂

MW 277.31

MP=73-75° C.

IR (KBr) 3441, 3051, 2954, 1731, 1582, 1556 cm⁻¹.

### Description B

### 3-Bromomethyl-2-phenyl-quinoline-4-carboxylic acid methyl ester

10 g (36 mmol) of 3-methyl-2-phenyl-quinoline-4-carboxylic acid methyl ester (compound of Description A) were dissolved in 500 ml of CH₃CN; 13 g (72 mmol) of N-bromosuccinimide were added and the reaction mixture was heated to reflux. After adding 1 g (4.1 mmol) of dibenzoylperoxide, the reaction was refluxed for 24 h; then additional 4 g (22.5 mmol) of N-bromosuccinimide and 0.5 g (2.0 mmol) of dibenzoylperoxide were added and the reaction was refluxed for 4 h. The solvent was evaporated in vacuo to dryness to yield 26.1 g of crude methyl 3-bromomethyl-2-phenylquinoline-4-carboxylate (theorical amount, 12.8 g) which was used in the following reaction without further purification.

C₁₈H₁₄BrNO₂

MW=356.23

### Description 1

### 3-(4-Fmoc-piperazin-1-ylmethyl)-2-phenyl-quinoline-4-carboxylic acid methyl ester

6.6 g (18.5 mmol) of crude 3-bromomethyl-2-phenyl-quinoline-4-carboxylic acid methyl ester (compound of Description B) were dissolved, under nitrogen atmosphere, in 100 ml of dry THF. The solution was cooled to 10° C. and 6.8 g (20 mmol) of Fmoc piperazine, dissolved in 50 ml of THF, were added dropwise. The reaction mixture was allowed to warm to room temperature and stirred overnight. Salts were filtered off and the filtrate was evaporated in vacuo to dryness, taken up with 2 N HCl and washed with EtOAc; the aqueous layer was basified with 10% NaOH and extracted with CH₂Cl₂. The organic layer was dried over Na₂SO₄, filtered and evaporated in vacuo to dryness to obtain a crude material. Flash chromatography on silica gel afforded 7.5 g (yield: 69%) of the title compound.

C₃₇H₃₃N₃O₄

MW=583.68

¹H NMR δ (DMSO-d₆): 1.99 (4H); 3.10 (4H); 3.62 (2H); 3.97 (3H); 4.20 (1H); 4.42 (2H); 7.18-7.40 (4Har); 7.45-7.92 (12Har); 8.09 (1Har) ppm.

### Description 2

### 3-(4-Fmoc-piperazin-1-ylmethyl)-2-phenyl-quinoline-4-carboxylic acid hydrochloride

7.5 g (13 mmol) of the ester of Description 1 were dissolved in 150 ml of 6 N HCl and refluxed for 1 h. Evaporation to dryness afforded 9.5 g of crude title compound, which was used in the following reaction without further purification.

C₃₆H₃₁N₃O₄.HCl

MW=606.12

¹H NMR δ (DMSO-d₆): 2.50 (4H); 3.32 (4H); 4.22 (2H); 4.23 (1H); 4.35 (2H); 6.50 (1Hexch with D₂O); 7.22-7.88 (14Har); 7.98 (1Har); 8.17 (2Har) ppm.

### Description 3

### 3-(4-Fmoc-piperazin-1-ylmethyl)-2-phenyl-quinoline-4-carboxylic acid ((S)-1-phenyl-propyl)-amide

5.35 g (8.3 mmol) of crude acid of Description 2 were dissolved in 100 ml of dry THF; 1.7 ml (12.5 mmol) of triethylamine (TEA) and 4.1 g (10.79 mmol) of O-benzotriazol-1-yl-N,N,N′,N′-tetramethyluroniumhexafluoro-phosphate (HBTU) were added and the reaction mixture was cooled at 0° C. 1.7 ml (12.5 mmol) of (S)-1-phenyl-propylamine, dissolved in 40 ml of dry CH₂Cl₂, were added dropwise and the reaction mixture was stirred at room temperature for 24 h and at 50° C. for 2 h. The solvent was evaporated in vacuo to dryness and the residue was taken up with EtOAc and washed with H₂O, 1 N NaOH and brine, dried over Na₂SO₄ and evaporated to dryness. Flash chromatography on silica gel afforded 3.2 g (56%) of the title compound.

C₄₅H₄₂N₄O₃

MW=686.86

¹H NMR δ (DMSO-d₆): 0.94 (3H); 1.40-2.18 (6H); 2.57-3.13 (4H); 3.50(2H); 4.21 (1H); 4.34 (2H); 5.08 (1H); 7.09-7.98 (21Har); 8.03 (1Har): 9.12 (1Hexch with D₂O) ppm.

### Description 4

### 3-(4-Fmoc-piperazin-1-ylmethyl)-2-phenyl-quinoline-4-carboxylic acid ((S)-1-cyclohexyl-ethyl)-amide

4.75 g (8.3 mmol) of crude acid of Description 2 were condensed on 1.65 ml (11 mmol) of (S)-1-cyclohexyl-ethylamine following the procedure of Description 3 affording, after flash chromatography on silica gel, 2.2 g (yield 43.9%) of the title compound.

C₄₄H₄₆N₄O₃

MW=678.87

¹H NMR δ (DMSO-d₆): 0.95 (3H); 1.68-4.00 (21H); 2.60 (3H); 5.08 (1H); 7.22-8.24 (13Har); 8.11 (1Har); 9.32 (1Hexch with D₂O); 10.82 (2Hexch with D₂O) ppm.

### Description 5

### 3-(4-Fmoc-piperazin-1-ylmethyl)-2-phenyl-quinoline-4-carboxylic acid ((S)-1-phenyl-ethyl)-amide

Synthesised starting from the compound of Description 2 and following the procedure of Description 3.

C₄₄H₄₀N₄O₃

MW=672.83

### Description 6

### 2-Phenyl-3-piperazin-1-ylmethyl-quinoline-4-carboxylic acid ((S)-1-phenyl-propyl)-amide

2.75 g (41 mmol) of the Fmoc protected derivative of Description 3 was reacted with 1.0 ml of piperidine in 100 ml acetonitrile, at room temperature for one night. The reaction mixture is concentrated to dryness and the residue was purified by flash chromatography on silicagel, affording 1.14 g (yield 60%) of the title compound.

C₃₀H₃₂N₄O

MW=464.61

¹H NMR δ (DMSO-d₆): 0.94 (3H); 1.57-2.08 (6H); 2.31 (4H); 3.36 (2H and 1Hexch with D₂O); 5.07 (1H); 7.13-7.94 (13Har); 8.01 (1Har); 9.17 (1Hexch with D₂O) ppm.

### Description 7

### 2-Phenyl-3-piperazin-1-ylmethyl-quinoline-4-carboxylic acid ((S)-1-cyclohexyl-ethyl)-amide

Synthesised starting from the compound of Description 4 and following the procedure of Description 6.

C₂₉H₃₆N₄O

MW=456.63

### Description 8

### 2-Phenyl-3-piperazin-1-ylmethyl-quinoline-4-carboxylic acid ((S)-1-phenyl-ethyl)-amide

Synthesised starting from the compound of Description 5 and following the procedure of Description 6.

C₂₉H₃₀N₄O

MW=450.58

### Description 9

### (3-Oxo-3-{4-[2-phenyl-4-((S)-1-phenyl-propylcarbamoyl)-quinolin-3-ylmethyl]-piperazin-1-yl}-propyl)-carbamic acid tert-butyl ester

1.0 g (2.15 mmol) of 2-Phenyl-3-piperazin-1-ylmethyl-quinoline-4-carboxylic acid ((S)-1-phenyl-propyl)-amide (compound of Description 6), 0.45 g (2.58 mmol) of N-BOC-β-alanine, 0.31 g (3.22 mmol) and 1.22 g (3.22 mmol) of HBTU were dissolved in 50 ml of CH₂Cl₂ and the mixture was stirred for 4 hours at room temperature. The solvent was evaporated in vacuo to dryness and the residue was taken up with EtOAc and washed three times with 0.1 N NaOH and brine, dried over Na₂SO₄ and evaporated to dryness affording 0.53 g of crude title compound, which was used in the following reaction without further purification.

C₃₈H₄₅N₅O₄

MW=635.80

IR: (KBr) 3287, 3971, 1710, 1644, 1531, 1170, 849 cm⁻¹

### Description 10

### 3-Methyl-2-phenyl-quinoline-4-carboxylic acid ((S)-1-cyclohexyl-ethyl)-amide

4-Carboxy-3-methyl-2-phenylquinoline (40 g, 0.152 mol) was suspended in CH₂Cl₂ (600 ml) and oxalyl chloride (6.6 ml, 0.311 mol) was added dropwise at 0° C. under magnetic stirring. After 15 min 2 drops of DMF were added. The reaction was vigorous with gas evolution. The mixture was stirred at room temperature until the solid was completely dissolved (about 2 h). The solution was evaporated. The crude material was redissolved in CH₂Cl₂ (150 ml) and slowly dropped into a sospension of K₂CO₃ (47 g) and (S)-1-cyclohexylethyl amine (29 ml, 0.196 mol) in CH₂Cl₂ (250 ml) maintaining the temperature between 10-15° C. The dark solution was left 1 h at room temperature. and 1 h refluxing. The organic phase was then washed with water, NaOH 1N, brine, dried over Na₂SO₄ and then evaporated under vacuum. The crude residue was triturated with AcOEt. After filtration 46.6 g of the title compound were obtained, mp=177-180° C. Yield: 82%

C₂₅H₂₈N₂O

MW=372.51

[α]_{D}=+21.77 (c=0.4 in MeOH).

### Description 11

### 3-Bromomethyl-2-phenyl-quinoline-4-carboxylic acid ((S)-1-cyclohexyl-ethyl)-amide

3-Methyl-2-phenyl-quinoline-4-carboxylic acid ((S)-1-cyclohexyl-ethyl)-amide (9.8 g, 26 mmol; compound prepared as in Description 10) and N-bromosuccinimmide (9.8 g, 55 mmol) were suspended in CCl₄ (100 ml) and warmed to incipient reflux. Dibenzoyl peroxide (about 300 mg) was carefully added portionwise and the solution was then refluxed for 2 h. The solvent was removed under vacuum and the residue was re-dissolved in CH₂Cl₂ (200 ml) and filtered. DCM was then evaporated and the residue was dissolved in AcOEt and washed with a saturated solution of NaHCO₃, brine, dried over Na₂SO₄, filtered and evaporated to give 6.9 g of the title compound as a white powder that were in the next step used without further purification, mp: 182-184° C. Yield: 58%

C₂₅H₂₇BrN₂O

MW=451.41

[α]_{D}=−5.76 (c=0.5% in CH₂Cl₂)

### Description 12

### N-BOC-piperazine

To a solution of piperazine (30 g, 0.35 mol) in water (370 ml) and tBuOH (420 ml), a solution of 4N NaOH (70 ml) was added. The mixture was cooled to 0° C. and then BOC₂O (38 g, 0.17 mol) was added portionwise. After stirring at room temperature for 45 minutes, tBuOH was evaporated under vacuum, the precipitate (diBOC-piperazine) was filtered and water was extracted with CH₂Cl₂. After drying over Na₂SO₄ the solvent was removed under vacuum to afford the title compound as a white solid (17 g, 91 mmol), mp=60-62° C. Yield: 54%

C₉H₁₈N₂O₂

MW=186.25

### Description 13

### 4-[4-((S)-1-Cyclohexyl-ethylcarbamoyl)-2-phenyl-quinolin-3-ylmethyl]-piperazine-1-carboxylic acid tert-butyl ester

A solution 3-bromomethyl-2-phenyl-quinoline-4-carboxylic acid ((S)-1-cyclohexyl-ethyl)-amide (10.4 g, 0.023 mol; compound prepared as in Description 11), BOC-piperazine (4.7 g, 0.025 mol; compound prepared as in Description 12) and diisopropylethylamine (DIEA) (8.5 ml, 0.049 mol) in THF (200 ml) was stirred at room temperature for 36 h. The solvent was evaporated under vacuum, the residue was then re-dissolved in ethyl acetate, washed with a saturated solution of aqueous citric acid and the organic phase dried over Na₂SO₄. The solvent was removed under vacuum and the residue (12 g) was directly used for the next step without further purification.

C₃₄H₄₄N₄O₃

MW=556.75

### Description 14

### 2-Phenyl-3-piperazin-1-ylmethyl-quinoline-4-carboxylic acid ((S)-1-cyclohexyl-ethyl)-amide

To a solution of 4-[4-((S)-1-cyclohexyl-ethylcarbamoyl)-2-phenyl-quinolin-3-ylmethyl]-piperazine-1-carboxylic acid tert-butyl ester (12.1 g, 21.7 mmol; compound prepared as in Description 13) in CH₂Cl₂ (90 ml), TFA (30 ml) was added dropwise at room temperature. Stirring was continued for additional 3 h. The solvent was removed under vacuum and the residue was made alkaline with 1N NaOH and extracted with ethyl acetate. The organic layer was dried over Na₂SO₄, filtered and evaporated to give after purification with flash cromatography (CH₂Cl₂/MeOH/NH₄OH 93:7:0.1) the title compound (9.5 g, 20.8 mmol), mp=116-118° C. Yield: 96%

C₂₉H₃₆N₄O

MW=456.63

[α]_{D}=+18.16 (c=1% in MeOH).

### Description 15

### 3-(4-Acryloyl-piperazin-1-ylmethyl)-2-phenyl-quinoline-4-carboxylic acid ((S)-1-cyclohexyl-ethyl)-amide

Acryloyl chloride (0.4 ml, 4.7 mmol) was added at 0° C. to a solution of 2-phenyl-3-piperazin-1-ylmethyl-quinoline-4-carboxylic acid ((S)-1-cyclohexyl-ethyl)-amide (2 g, 4.4 mmol; compound prepared as in Description 14) in 30 ml of dry THF. Then TEA (0.7 ml, 4.7 mmol) in THF (5 ml) was added dropwise. The reaction was warmed to room temperature and then stirred for additional 4 h. The solvent was removed under vacuum. The residue was dissolved in AcOEt and washed with 2N NaOH, with H₂O and dried over Na₂SO₄. The solvent was evaporated to dryness and the crude compound was triturated with diisopropyl ether affording 2 g of the title compounds (yield: 89%).

C₃₂H₃₈N₄O₂

MW: 510.68

### Example 1

### (−)-(S)-N-(1-Phenylpropyl)-3-[4-(3-aminopropionyl)piperazin-1-yl]methyl-2-phenylquinoline-4-carboxamide dihydrochloride

0.2 g of (3-Oxo-3-{4-[2-phenyl-4-((S)-1-phenyl-propylcarbamoyl)-quinolin-3-ylmethyl]-piperazin-1-yl}-propyl)-carbamic acid tert-butyl ester (compound of Description 9) were dissolved in 10 ml of MeOH and 10 ml of a 30% solution of HCl in Et₂O. The solution was stirred at room temperature for 6 hours then the solvent was evaporated in vacuo to dryness. The residue was taken up with Et₂O and evaporated in vacuo to dryness for three times. The residue was triturated with Et₂O, collected by suction and dried at 50° C. under mechanical vacuum to afford 0.15 g of the title compound as a yellow powder.

C₃₃H₃₇N₅O₂.2(HCl)

MW=608.61

IR: (KBr) 3420, 3167, 2967, 1654, 1542 cm⁻¹ cm⁻¹

### Example 12

### 3-(1-{4-[4-((S)-1-Cyclohexyl-ethylcarbamoyl)-2-phenyl-quinolin-3-ylmethyl]-piperazin-1-yl}-methanoyl)-pyrazine-2-carboxylic acid

3 g (6.6 mmol) of 2-phenyl-3-piperazin-1-ylmethyl-quinoline-4-carboxylic acid ((S)-1-cyclohexyl-ethyl)-amide (compound of Description 7) and 1.1 g (7.2 mmol) of 2,3-pyrazinedicarboxylic anhydride were dissolved in 100 ml of THF and the solution was refluxed for 12 hours. The solvent was removed under vacuum and the residue was triturated with diisopropyl ether (50 ml), collected by suction and dried at 40° C. under mechanical vacuum to afford 3.8 g (yield: 95%) of the title compound.

C₃₅H₃₈N₆O₄

MW=606.72

M.P.=162-165° C.

IR: (KBr) 2924, 1633, 1461, 1377 cm⁻¹.

### Example 16

### 3-{4-[4-((S)-1-Cyclohexyl-ethylcarbamoyl)-2-phenyl-quinolin-3-ylmethyl]-piperazin-1-yl}-3-oxo-2-phenyl-propionic acid ethyl ester

A solution of 0.24 g (1.05 mmol) of 2-chlorocarbonyl-2-phenyl-acetic acid ethyl ester (RN 54635-33-5) in 2 ml of CH₂Cl₂ was added to an ice cooled solution of 0.4 g (0.87 mmol) of 2-phenyl-3-piperazin-1-ylmethyl-quinoline-4-carboxylic acid ((S)-1-cyclohexyl-ethyl)-amide (compound of Description 7), 0.11 g (1.05 mmol) of triethyl amine in 5 ml CH₂Cl₂ stabilized by amylene and the mixture was stirred at room temperature for 3 h. The solvent was concentrated and the residue dissolved in AcOEt. The organic phase was washed twice with water and dried over MgSO₄. After concentration of the solvent the residue was purified by flash chromatography on 40 g silicagel (eluent, heptane/AcOEt: 55/45) affording 0.25 g of a pure fraction and 0.21 g of impure fraction. This second fraction was purified by flash chromatography in the same conditions affording a second pure fraction of 0.13 g. Total: 0.25 g (67.5%) of the title compound as white crystals.

C₄₀H₄₆N₄O₄

MW=646.83

M.P.=125-129° C.

### Example 17

### 3-{4-[4-((S)-1-Cyclohexyl-ethylcarbamoyl)-2-phenyl-quinolin-3-ylmethyl]-piperazin-1-yl}-3-oxo-2-phenyl-propionic acid sodium salt

A mixture of 0.25 g (3.9 mmol) of 3-{4-[4-((S)-1-cyclohexyl-ethylcarbamoyl)-2-phenyl-quinolin-3-ylmethyl]-piperazin-1-yl}-3-oxo-2-phenyl-propionic acid ethyl ester (compound of Example 16), 0.390 g (3.9 mmol) of 1 N aqueous sodium hydroxide and 5 ml of ethanol was stirred for 3 h at room temperature. The solvent was concentrated and the residue suspended in diethyl ether. The solid was filtered and washed three times with ether affording 0.23 mg of crude sodium salt. The crude compound was stirred with a small amount of AcOEt and the precipitate washed with small fractions of AcOEt affording 0.16 g of the title compound as white crystals

C₃₈H₄₁N₄O₄Na

MW=640.76

M.P.: 152-157° C.

### Example 19

### ((S)-N-1-Cyclohexylethyl)-2-phenyl-3-(4-phenylcarbamoylpiperazin-1-ylmethyl)quinoline-4-carboxamide

To a solution of 0.2 g (0.4 mmol) of 2-phenyl-3-piperazin-1-ylmethyl-quinoline-4-carboxylic acid ((S)-1-cyclohexyl-ethyl)-amide (compound of Description 7), in 10 ml of CH₂Cl₂ were added 0.047 ml (0.4 mmol) of phenylisocyanat. The mixture was stirred for 4 hours and then the solvent was removed under vacuum and the residue was purified by flash cromatography (eluent: AcOEt:hexane 1:1) to afford 0.112 g of the title compound (yield: 49%.)

C₃₆H₄₁N₅O₂

MW: 575.753

M.P.: 168-170° C.

IR: (KBr) 2921, 1633, 1456, 1377, 1238 cm⁻¹.

### Example 20

### ((S)-N-1-Cyclohexylethyl)-2-phenyl-3-(4-carbamoylpiperazin-1-ylmethyl) quinoline-4-carboxamide

To a solution of 0.2 g (0.4 mmol) of 2-phenyl-3-piperazin-1-ylmethyl-quinoline-4-carboxylic acid ((S)-1-cyclohexyl-ethyl)-amide (compound of Description 7), in 3 ml of 1:20 mixture of AcOH:H₂O, were added 0.026 g (0.4 mmol) of sodium isocyanate in 2 ml of water. The mixture was stirred for 3 hours and then the white powder was filtered and washed with H₂O to 0.015 mg of the title compound (Yield: 8%).

C₃₀H₃₇N₅O₂

MW: 499.66

### Example 21

### 3-[4-(3-Amino-propanoyl)-piperazin-1-ylmethyl]-2-phenyl-quinoline-4-carboxylic acid ((S)-1-cyclohexyl-ethyl)-amide

A solution of 2-phenyl-3-piperazin-1-ylmethyl-quinoline-4-carboxylic acid ((S)-1-cyclohexyl-ethyl)-amide (2 g, 4.4 mmol; compound of Description 14), 1.1 g (5.8 mmol) of 3-tert-butoxycarbonylaminopropionic acid, 1.2 g (5.8 mmol) of DCC and 0.7 g (5.8 mmol) of DMAP in 60 ml of CH₂Cl₂ was stirred for 24 h at room temperature. The solid was filtered and the filtrate was evaporated to dryness. The residue was dissolved in AcOEt, washed with a 10% NaCl solution and dried over MgSO₄. After solvent evaporation, the crude product was dissolved in 60 ml of CH₂Cl₂ and 3 ml of TFA were added. The red solution was stirred at room temperature overnight; then the solvent and the excess of TFA were removed under vacuum. The residue was dissolved in H₂O and washed 2 times with Et₂O. The water extract was made alkaline by addition of 2N NaOH and the product was extracted with AcOEt. The solvent was evaporated to dryness and the residue was purified by flash chromatography (eluent CH₂Cl₂: MeOH 93:7) to afford 0.7 g of the title compound (yield: 30%).

C₃₂H₄₁N₅O₂

MW: 527.709

### Example 22

### 3-[4-(3-Ethylamino-propanoyl)-piperazin-1-ylmethyl]-2-phenyl-quinoline-4-carboxylic acid ((S)-1-cyclohexyl-ethyl)-amide

Ethylamine solution (70%, 4 ml) was added to 3-(4-acryloyl-piperazin-1-ylmethyl)-2-phenyl-quinoline-4-carboxylic acid ((S)-1-cyclohexyl-ethyl)-amide (0.6 g, 1.2 mmol; compound of Description 15) in 20 ml of CH₂Cl₂. The mixture was stirred overnight at room temperature and then the solvent and the excess of amine were removed under vacuum. The residue was purified by flash chromatography (eluent CH₂Cl₂:MeOH:NH₄OH 93:7:0.2) to afford 0.4 g of the title compound (yield 60%).

C₃₄H₄₅N₅O₂

MW: 555.763

### Example 23

### 2-Phenyl-3-[4-(3-pyrrolidin-1-yl-propanoyl)-piperazin-1-ylmethyl]-quinoline-4-carboxylic acid ((S)-1-cyclohexyl-ethyl)-amide

A solution of 3-(4-acryloyl-piperazin-1-ylmethyl)-2-phenyl-quinoline-4-carboxylic acid ((S)-1-cyclohexyl-ethyl)-amide (0.3 g, 0.6 mmol; compound of Description 15) and pyrrolidine (6 ml) in 10 ml of CH₂Cl₂ was stirred overnight at room temperature. The excess of pyrrolidine and the solvent were removed under vacuum and the residue was triturated with diisopropyl ether affording 0.2 g of the title compound (yield 57%).

C₃₆H₄₇N₅O₂

MW: 581.800

### Example 24

### 2-Phenyl-3-[4-(3-piperidin-1-yl-propanoyl)-piperazin-1-ylmethyl]-quinoline-4-carboxylic acid ((S)-1-cyclohexylethyl)-amide

2-Phenyl-3-(piperazin-1-ylmethyl)-quinoline-4-carboxylic acid ((S)-1-cyclohexyl-ethyl)-amide (0.5 g, 1.1 mmol; compound of Description 14) and 3-piperidin-1-yl-propionic acid (0.22 g, 1.4 mmol) were dissolved in 40 ml of CH₂Cl₂. Then DCC (0.3 g, 1.4 mmol) and DMAP (0.2 g, 1.4 mmol) were added. The suspension was stirred overnight at room temperature. The solid was filtered and the organic solvent was removed under vacuum. The residue was dissolved in AcOEt, washed with water and dried over Na₂SO₄. After solvent evaporation, the crude compound was purified by flash chromatography (eluent CH₂Cl₂:MeOH 93:7) affording 0.4 g of the title compound (yield 61%).

C₃₇H₄₉N₅O₂

MW: 595.827

TABLE 1

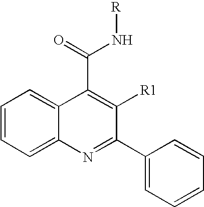

| | | | | | Melting | |
|---|---|---|---|---|---|---|
| | | | Molecular | Molecular | Point | [a]_{D}²⁰ |
| Ex. | R | R₁ | Formula | Weight | (° C.) | (c = 0.5, MeOH) |
| 1 | 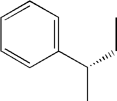 | 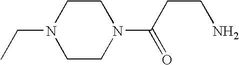 | C₃₃H₃₇N₅O₂. 2HCl | 608.61 | 196-197 | —25.04(c = 0.33) |
| 2 | 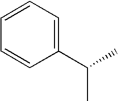 | 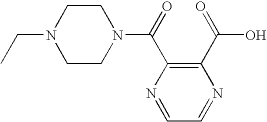 | C₃₆H₃₃N₅O₄ | 600.68 | 190-195 dec | |
| 3 | 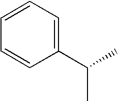 | 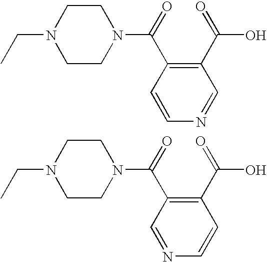 | C₃₅H₃₂N₆O₄ | 599.69 | 185-195 dec | |
| 4 | 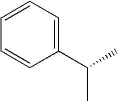 | 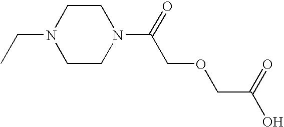 | C₃₆H₃₃N₅O₄ | 566.66 | 122-128 | |
| 5 | 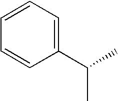 | 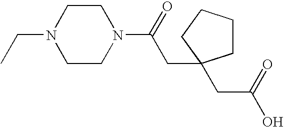 | C₃₃H₃₄N₄O₅ | 618.774 | | |
| 6 | 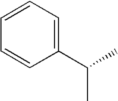 | 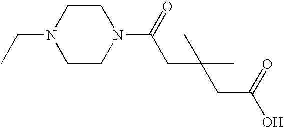 | C₃₆H₄₀N₄O₄ | 592.74 | 213-215 | |
| 7 | 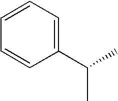 | 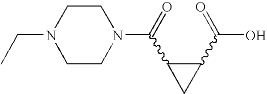 | C₃₄H₃₄N₄O₄ | 562.67 | 135-145 | |
| 8 | 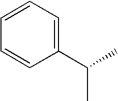 | 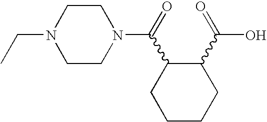 | C₃₇H₄₀N₄O₄ | 604.75 | 130-140 | |
| 9 | 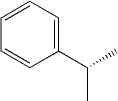 | 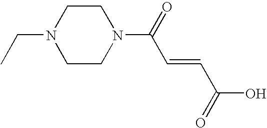 | C₃₃H₃₂N₄O₄ | 548.64 | 214-215 | |
| 10 | 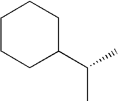 | 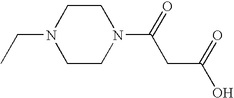 | C₃₂H₃₈N₄O₄ | 542.68 | 150-155 | |
| 11 | 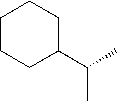 | 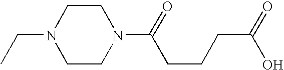 | C₃₄H₄₂N₄O₄ | 570.73 | 125-130 | |
| 12 | 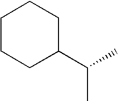 | 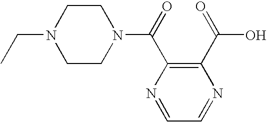 | C₃₅H₃₈N₆O₄ | 606.72 | 162-165 | +9.712 (c = 1) |
| 13 | 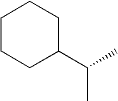 | 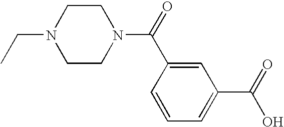 | C₃₇H₄₀N₄O₄ | 604.75 | 179-180 | |
| 14 | 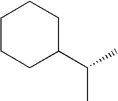 | 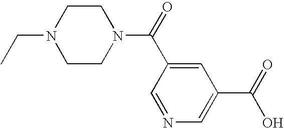 | C₃₆H₃₉N₅O₄ | 605.74 | 250-255 dec | |
| 15 | 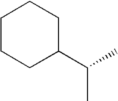 | 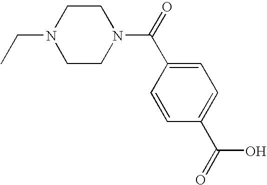 | C₃₇H₄₀N₄O₄ | 604.75 | 185-188 | |
| 16 | 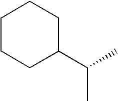 | 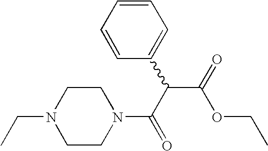 | C₄₀H₄₆N₄O₄ | 646.83 | 125-129 | |
| 17 | 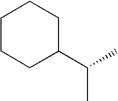 | 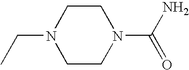 | C₃₈H₄₁N₄O₄Na | 640.76 | 152-157 | |
| 18 | 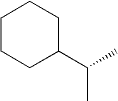 | 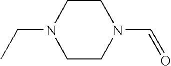 | C₃₀H₃₆N₄O₂ | 484.64 | 116-119 | |
| 19 | 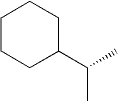 | 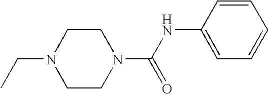 | C₃₆H₄₁N₅O₂ | 575.75 | 168-170 | +12.62 (c = 0.1) |
| 20 | 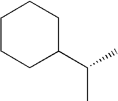 | 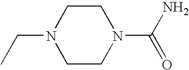 | C₃₀H₃₇N₅O₂ | 499.66 | | |
| 21 | 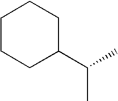 | 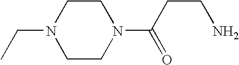 | C₃₂H₄₁N₅O₂ | 527.709 | 164-166 | +6.84 |
| 22 | 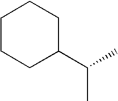 | 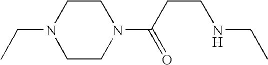 | C₃₄H₄₅N₅O₂ | 555.763 | 125 | +4.5 |
| 23 | 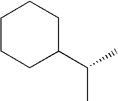 | 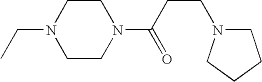 | C₃₆H₄₇N₅O₂ | 581.800 | | −16.54 (c = 0.1) |
| 24 | 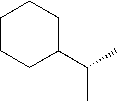 | 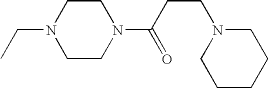 | C₃₇H₄₉N₅O₂ | 595.827 | 120-122 | +11.08 |

TABLE 2
¹H NMR data of compounds of Examples of Table 1

| Ex. | 300MHz¹H NMR (Solvent) ppm |
|---|---|
| 1 | (DMSO, 343K, TFA): 9.50(d br, 1H); 8.11(d, 1H); 7.95-7.72(m, 3H); |
| | 7.71-7.47(m, 7H); 7.41(dd, 2H); 7.31(dd, 1H); 5.13(dt, 1H); 4.05(s br; 2H); |
| | 3.31(m, 4H); 2.98(m, 2H); 2.61(t, 2H); 2.41(m, 4H); 2.08-1.79(m, 2H); |
| | 0.98(t, 3H). |
| 2 | (CDCl3): 1.71(d, 3H); 1.90-2.48(m, 4H); 2.78(m, 2H); 3.45(m, 2H); |
| | 4.05(m, 2H); 4.65(br, 1H); 5.55(m, 1H); 6.95-7.60(m, 11Har); 7.68(t, 1Har); |
| | 7.85(1Har); 8.03(1Har); 8.53(m, 2Har); 8.80(br, 1H)ppm. |
| 3 | (CDCl3): 1.90-3.10(m, 10H); 3.65-4.35(m, 4H); 5.55(m, 1H); |
| | 6.85-7.87(m, 16Har); 8.82(m, 1Har); 8.57(m, 1H)ppm. |
| 4 | (CDCl3): 1.71(d, 3H); 1.81-2.20(m, 4H); 2.83(m, 2H); 3.19(m, 2H); |
| | 3.67(s, 2H); 4.09(s, 2H); 4.19(s, 2H); 4.85(br, 1H); 5.54(m, 1H); |
| | 7.30-7.53(m, 11H); 7.60(t, 1Har); 7.76(t, 1Har); 7.96(d, 1Har); 8.14(d, 1Har)ppm. |
| 5 | (DMSO-d6): 1.52(m, 11H); 1.64-2.23(m, 4H); 2.40(m, 4H); |
| | 2.82-3.73(m, 8H); 5.81(m, 1H); 7.17-7.98(12Har); 8.04(d, 1Har); |
| | 9.18(d, br, 1H)ppm. |
| 6 | (CDCl3): 1.00(s, 6H); 1.60(br, 1H); 1.72(d, 3H); 2.03(m, 4H); 2.32(m, 4H); |
| | 3.04(m, 2H); 3.22(m, 2H); 3.65(s, 2H); 5.53(m, 1H); 7.30-7.55(m, 11H); |
| | 7.60(t, 1Har); 7.76(t, 1Har); 7.98(d, 1Har); 8.14(d, 1Har)ppm. |
| 7 | (CDCl3): 1.17-1.60(m, 2H); 1.70(d, 3H); 1.79-2.28(m, 7H); 3.14(m, 4H); |
| | 3.67(s, 2H); 5.55(m, 1H); 7.18-7.82(13H); 7.95(m, 1H); 8.13(d, 1Har)ppm. |
| 8 | (CDCl3): 1.15-2.20(m, 12H); 1.71(d, 3H); 2.30(m, 1H); 2.55-3.33(m, 6H); |
| | 3.64(s, 2H); 5.52(m, 1H); 7.25-7.65(m, 12H); 7.75(t, 1H); 7.98(d, 1H); |
| | 8.14(d, 1H)ppm. |
| 9 | (DMSO-d6): 1.48(d, 3H); 1.59-2.32(m, br, 4H); 2.78-3.55(m, br, 7H); |
| | 5.28(m, 1H); 6.43(d, 1H); 6.95(d, 1H); 7.17-7.97(m, 13H); 8.02(d, 1H); |
| | 9.22(d, 1H)ppm. |
| 10 | (CDCl3): 0.92-1.38(m, 5H); 1.27(d, 3H); 1.48(m, 1H); 1.60-1.97(m, 6H); |
| | 2.24(m, 4H); 3.02-3.32(m, 4H); 3.42(m, 2H); 3.72(s, 2H); 4.25(m, 1H); |
| | 6.40(br, 1H); 7.34-7.57(m, 5Har); 7.60(t, 1Har); 7.76(t, 1Har); 7.94(d, 1Har); |
| | 8.15(d, 1Har)ppm. |
| 11 | (CDCl3): 0.95-2.02(m, 16H); 1.28(d, 3H); 2.18(m, 4H); 2.25-2.50(m, 4H); |
| | 3.22(m, 2H); 3.38(m, 2H); 3.70(m, 1H); 6.95(br, 1H); 7.33-7.55(m, 5Har); |
| | 7.60(t, 1Har); 7.75(t, 1Har); 7.99(d, 1Har); 8.14(d, 1Har)ppm. |
| 12 | (DMSO-d6, as sodium salt, 343K): 8.42(s br, 1H); 8.36(d, 1H); 8.26(d, |
| | 1H); 8.01(d, 1H); 7.86(d, 1H); 7.75(dd, 1H); 7.62(dd, 1H); 7.56(d, 2H); |
| | 7.51-7.40(m, 3H); 4.04(dq, 1H); 3.63(s, 2H); 3.29(m, 2H); 2.81(m, 2H); |
| | 2.16(m, 4H); 1.87-1.60(m, 5H); |
| 13 | (DMSO-d6): 0.85-1.94(m, 14H); 2.12(m, 4H); 2.80-3.50(m, 5H); 3.58(s, 2H); |
| | 4.00(m, 1H); 7.25-8.15(m, 13Har); 8.56(br, 1H)ppm. |
| 14 | (DMSO-d6): 0.95-1.35(m, 5H); 1.15(d, 3H); 1.45(m, 1H); 1.55-1.92(m, 5H); |
| | 2.13(m, 4H); 2.91-3.50(m, 5H); 3.60(s, 2H); 4.02(m, 1H); 7.35-7.90(m, 8Har); |
| | 7.98-8.12(m, 2Har); 8.47(s, 1Har); 8.55(br, 1H); 9.01(s, 1Har)ppm. |
| 15 | (CDCl3): 0.95-1.39(m, 5H); 1.28(d, 3H); 1.48(m, 1H); 1.62-1.97(m, 5H); |
| | 2.19(m, 4H); 3.15(m, 2H); 3.53(m, 2H); 3.75(s; 2H); 4.27(m, 1H); |
| | 4.70(br, 1H); 6.75(br, 1H); 7.31-7.67(m, 8Har); 7.75(t, 1Har); 7.97(d, 1Har); |
| | 8.04(d, 2Har); 8.15(d, 1Har)ppm. |
| 16 | (DMSO-d₆)□ 0.95-1.53(12H); 1.60-1.95(5H); 2.00-2.35(m, 4H); |
| | 3.09(m, 2H); 3.42(m, 2H); 3.65(s, 2H); 4.08-4.34(m, 3H); 4.70(s, 1H); |
| | 6.88(br, 1H); 7.15-7.52(10Har); 7.52(td, 1Har); 7.73(td, 1Har); |
| | 7.97(dd, 1Har); 8.12(dd, 1Har)ppm. |
| 17 | (DMSO-d₆)□ 0.90-2.20(m, 18H); 2.78-3.75(m, 6H); 4.15(m, 1H); 4.53(s1H); |
| | 6.80-7.18(m, 6Har); 7.32-7.52(m, 5Har); 7.57(t, 1Har); 7.72(t, 1Har); |
| | 7.95(d, 1Har); 8.11(d, 1Har)ppm |
| 18 | (CDCl3): 1.00-1.98(m, 14H); 2.18(m, 4H); 3.12(m, 2H); 3.30(m, 2H); |
| | 3.74(s, 2H); 4.29(m, 1H); 7.35-7.68(m, 7H); 7.76(t, 1Har); 7.90(s, 1H); |
| | 8.98(d, 1Har); 8.15(d, 1Har)ppm. |
| 19 | (DMSO-d₆, 343K): 8.29 d br, 1H); 8.17(s br, 1H); 8.04(d, 1H); 7.87(d, |
| | 1H); 7.77(dd, 1H); 7.64(dd,, 1H); 7.59(dd, 2H); 7.52-7.44(m, 3H); |
| | 7.40(dd, 2H); 7.19(dd, 2H); 6.91(dd, 1H); 4.06(dt, 1H); 3.64(s, 2H); |
| | 3.19(m, 4H); 2.16(m, 4H); 1.88-1.72(m, 4H); 1.63(m, 1H); 1.51(m, 1H); |
| | 1.27-1.05(m, 5H); 1.21(d, 3H). |
| 20 | (DMSO-d₆, 343K): 8.28(d br, 1H); 8.02(d, 1H); 7.86(d, 1H); 7.76(dd, 1H); |
| | 7.63(dd, 1H); 7.57(m 2H); 7.517.41(m, 3H); 5.54(s br, 2H); 4.04(dt, 1H); |
| | 3.60(s, 2H); 3.01(m, 4H); 2.07(m, 4H); 1.88-1.72(m, 4H); 1.65(m, 1H); |
| | 1.51(m, 1H); 1.32-1.05(m, 5H); 1.20(d, 3H). |
| 21 | (DMSO-d₆, 343K)δ: 8.26(d br, 1H); 8.02(d, 1H); 7.87(d, 1H); 7.77(dd, |
| | 1H); 7.63(dd, 1H); 7.57(m, 2H); 7.51-7.41(m,, 3H); 4.04(m, 1H); 3.62(s, |
| | 2H); 3.18(m, 4H); 3.01(s br, 1H); 2.72(t, 2H); 2.29(t, 2H); 2.11(m, 4H); |
| | 1.88-1.71(m, 4H); 1.65(m, 1H); 1.50(m, 1H); 1.30-1.03(m, 5H); 1.20(d, |
| | 3H). |
| 22 | (DMSO-d₆, 343K)δ: 8.27(d br, 1H); 8.03(d, 1H); 7.86(d, 1H); 7.77(dd, |
| | 1H); 7.63(dd, 1H); 7.57(m, 2H); 7.51-7.41(m, 3H); 4.04(m, 1H); 3.62(s, |
| | 2H); 3.18 m, 4H); 2.67(t, 2H); 2.51(q, 2H); 2.34(t, 2H); 2.11(m, 4H); |
| | 1.88-1.71(m, 4H); 1.65(m, 1H); 1.50(m, 1H); 1.30-1.03(m, 5H); 1.20(d, |
| | 3H); 0.97(t, 3H). |
| 23 | (DMSO-d₆, 343K)δ: 8.28(d br, 1H); 8.03(d, 1H); 7.86(d, 1H); 7.77(dd, |
| | 1H); 7.63(dd, 1H); 7.57(m, 2H); 7.51-7.41(m,, 3H); 4.04(m, 1H); 3.61(s, |
| | 2H); 3.18(m, 4H); 2.59(t, 2H); 2.42(m, 4H); 2.37(t, 2H); 2.11(m, 4H); |
| | 1.88-1.69(m, 4H); 1.65(m, 5H); 1.50(m, 1H); 1.30-1.03(m, 5H); 1.20(d, |
| | 3H). |
| 24 | (DMSO-d₆, 343K)δ: 8.27(d br, 1H); 8.03(d, 1H); 7.86(d, 1H); 7.77(dd, |
| | 1H); 7.63(dd, 1H); 7.47(m, 2H); 7.51-7.41(m,, 3H); 4.04(m, 1H); 3.62(s, |
| | 2H); 3.18(m, 4H); 2.50(t, 2H); 2.34(m, 6H); 2.11(m, 4H); 1.88-1.59(m, |
| | 5H); 1.58-1.31(m, 5H); 1.30-1.03(m, 7H); 1.20(d, 3H). |

TABLE 3
Mass Spectra data of compounds of Examples of Table 1

| | m/z |
|---|---|
| | (ESI POS; AQA; solvent: methanol/spray 3 kV/ |
| Ex. | skimmer: 20 V/probe 135 C.) |
| 1 | 536(MH+); 465; 268.7(MHH++) |
| 2 | 607(MH+) |
| 19 | 576(MH+) |
| 21 | 528(MH+); 264.5(MHH++) |
| 22 | 556(MH+) |
| 23 | 582(MH+) |
| 24 | 596(MH+); 373; 220 |

TABLE 4
Chemical names of parent compounds of Examples of Table 1 (names generated by
Beilstein's Autonom)

| Example | Chemical name |
|---|---|
| 1 | (−)-(S)-N-(1-Phenylpropyl)-3-[4-(3-aminopropionyl)piperazin-1- |
| | yl]methyl-2-phenylquinoline-4-carboxamide dihydrochloride |
| 2 | 3-(1-{4-[2-Phenyl-4-((S)-1-phenyl-ethylcarbamoyl)-quinolin-3- |
| | ylmethyl]-piperazin-1-yl}-methanoyl)-pyrazine-2-carboxylic acid |
| 3 | 1:1 Mixture of 4-(1-{4-[2-phenyl-4-((S)-1-phenyl-ethylcarbamoyl)- |
| | quinolin-3-ylmethyl]-piperazin-1-yl}-methanoyl)-nicotinic acid |
| | and 3-(1-{4-[2-phenyl-4-((S)-1-phenyl-ethylcarbamoyl)-quinolin-3- |
| | ylmethyl]-piperazin-1-yl}-methanoyl)-isonicotinic acid |
| 4 | (2-Oxo-2-{4-[2-phenyl-4-((S)-1-phenyl-ethylcarbamoyl)-quinolin- |
| | 3-ylmethyl]-piperazin-1-yl}-ethoxy)-acetic acid |
| 5 | [1-(2-Oxo-2-{4-[2-phenyl-4-((S)-1-phenyl-ethylcarbamoyl)- |
| | quinolin-3-ylmethyl]-piperazin-1-yl}-ethyl)-cyclopentyl]-acetic acid |
| 6 | 3,3-Dimethyl-5-oxo-5-{4-[2-phenyl-4-((S)-1-phenyl- |
| | ethylcarbamoyl)-quinolin-3-ylmethyl]-piperazin-1-yl}-pentanoic acid |
| 7 | Trans 2-(1-{4-[2-Phenyl-4-((S)-1-phenyl-ethylcarbamoyl)-quinolin- |
| | 3-ylmethyl]-piperazin-1-yl}-methanoyl)-cyclopropanecarboxylic |
| | acid |
| 8 | Cis 2-(1-{4-[2-Phenyl-4-((S)-1-phenyl-ethylcarbamoyl)-quinolin-3- |
| | ylmethyl]-piperazin-1-yl}-methanoyl)-cyclohexanecarboxylic acid |
| 9 | (E)-4-Oxo-4-{4-[2-phenyl-4-((S)-1-phenyl-ethylcarbamoyl)- |
| | quinolin-3-ylmethyl]-piperazin-1-yl}-but-2-enoic acid |
| 10 | 3-{4-[4-((S)-1-Cyclohexyl-ethylcarbamoyl)-2-phenyl-quinolin-3- |
| | ylmethyl]-piperazin-1-yl}-3-oxo-propionic acid |
| 11 | 5-{4-[4-((S)-1-Cyclohexyl-ethylcarbamoyl)-2-phenyl-quinolin-3- |
| | ylmethyl]-piperazin-1-yl}-5-oxo-pentanoic acid |
| 12 | 3-(1-{4-[4-((S)-1-Cyclohexyl-ethylcarbamoyl)-2-phenyl-quinolin-3- |
| | ylmethyl]-piperazin-1-yl}-methanoyl)-pyrazine-2-carboxylic acid |
| 13 | 3-(1-{4-[4-((S)-1-Cyclohexyl-ethylcarbamoyl)-2-phenyl-quinolin-3- |
| | ylmethyl]-piperazin-1-yl}-methanoyl)-benzoic acid |
| 14 | 5-(1-{4-[4-((S)-1-Cyclohexyl-ethylcarbamoyl)-2-phenyl-quinolin-3- |
| | ylmethyl]-piperazin-1-yl}-methanoyl)-nicotinic acid |
| 15 | 4-(1-{4-[4-((S)-1-Cyclohexyl-ethylcarbamoyl)-2-phenyl-quinolin-3- |
| | ylmethyl]-piperazin-1-yl}-methanoyl)-benzoic acid |
| 16 | 3-{4-[4-((S)-1-Cyclohexyl-ethylcarbamoyl)-2-phenyl-quinolin-3- |
| | ylmethyl]-piperazin-1-yl}-3-oxo-2-phenyl-propionic acid ethyl ester |
| 17 | 3-{4-[4-((S)-1-Cyclohexyl-ethylcarbamoyl)-2-phenyl-quinolin-3- |
| | ylmethyl]-piperazin-1-yl}-3-oxo-2-phenyl-propionic acid sodium salt |
| 18 | 3-(4-Formyl-piperazin-1-ylmethyl)-2-phenyl-quinoline-4-carboxylic acid ((S)-1- |
| | cyclohexyl-ethyl)-amide |
| 19 | ((S)-N-1-Cyclohexylethyl)-2-phenyl-3-(4- |
| | phenylcarbamoylpiperazin-1-ylmethyl)quinoline-4-carboxamide |
| 20 | ((S)-N-1-Cyclohexylethyl)-2-phenyl-3-(4-carbamoylpiperazin-1- |
| | ylmethyl)quinoline-4-carboxamide |
| 21 | 3-[4-(3-Amino-propanoyl)-piperazin-1-ylmethyl]-2-phenyl- |
| | quinoline-4-carboxylic acid ((S)-1-cyclohexyl-ethyl)-amide |
| 22 | 3-[4-(3-Ethylamino-propanoyl)-piperazin-1-ylmethyl]-2-phenyl- |
| | quinoline-4-carboxylic acid ((S)-1-cyclohexyl-ethyl)-amide |
| 23 | 2-Phenyl-3-[4-(3-pyrrolidin-1-yl-propanoyl)-piperazin-1-ylmethyl]- |
| | quinoline-4-carboxylic acid ((S)-1-cyclohexyl-ethyl)-amide |
| 24 | 2-Phenyl-3-[4-(3-piperidin-1-yl-propanoyl)-piperazin-1-ylmethyl]- |
| | quinoline-4-carboxylic acid ((S)-1-cyclohexyl-ethyl)-amide |

1. A compound of formula (I) below or a pharmaceutically acceptable salt or hydrate thereof: 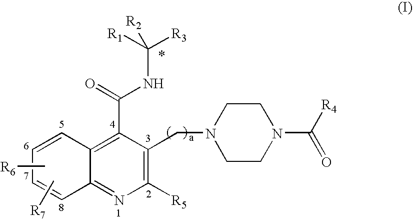
wherein:
R₁ is H or alkyl;
R₂ is aryl, cycloalkyl or heteroaryl;
R₃ is H or C₁₋₃ alkyl, optionally substituted by one or more fluorines;
R₄ is H, R₈NR₉R₁₀, R₁₁R₁₃ or R₁₁R₁₂R₁₃;
R₈ is a single bond or alkyl;
R₉ and R₁₀ are selected independently from H, alkyl, cycloalkyl or cycloalkylC₁₋₃alkyl, aryl or arylC₁₋₃alkyl, or R₉ and R₁₀ together with the nitrogen atom to which they are attached form a saturated or unsaturated heterocyclic ring which is optionally substituted by one or more fluorines;
R₁₁ is alkyl, alkenyl, aryl, heteroaryl, a saturated or unsaturated carbon ring including one or more heteroatoms selected from N, O and S, cycloalkyl, arylalkyl or cycloalkylalkyl, optionally substituted one or more times by C₁₋₃ alkyl, phenyl and/or phenylC₁₋₃alkyl;
R₁₂ is alkyl or alkoxy, optionally substituted one or more times by C₁₋₃ alkyl and/or by phenyl;
R₁₃ is H or COO R₁₄;
R₁₄ is H or alkyl;
R₅ is branched or linear alkyl, cycloalkyl, cycloalkylalkyl, aryl, arylalkyl, or a single or fused ring aromatic heterocyclic group;
R₆ represents H or up to three substituents independently selected from the list consisting of: alkyl, alkenyl, aryl, alkoxy, hydroxy, halogen, nitro, cyano, carboxy, carboxamido, sulphonamido, alkoxycarbonyl, trifluoromethyl, acyloxy, amino or mono- or di-alkylamino;
R₇ is H or halo;
a is 1-6; and
any of R₂, R₅, R₈, R₉, R₁₀, R₁₁, R₁₂ and R₁₄ may optionally be substituted one or more times by halo, hydroxy, amino, cyano, nitro, carboxy or oxo;
subject to the proviso that said compound is not a compound of formula (I) wherein R₇ represents H, R₆ represents H, R₅ represents phenyl, and R₁, R₂, R₃, R₄, and a are one of the following combinations:

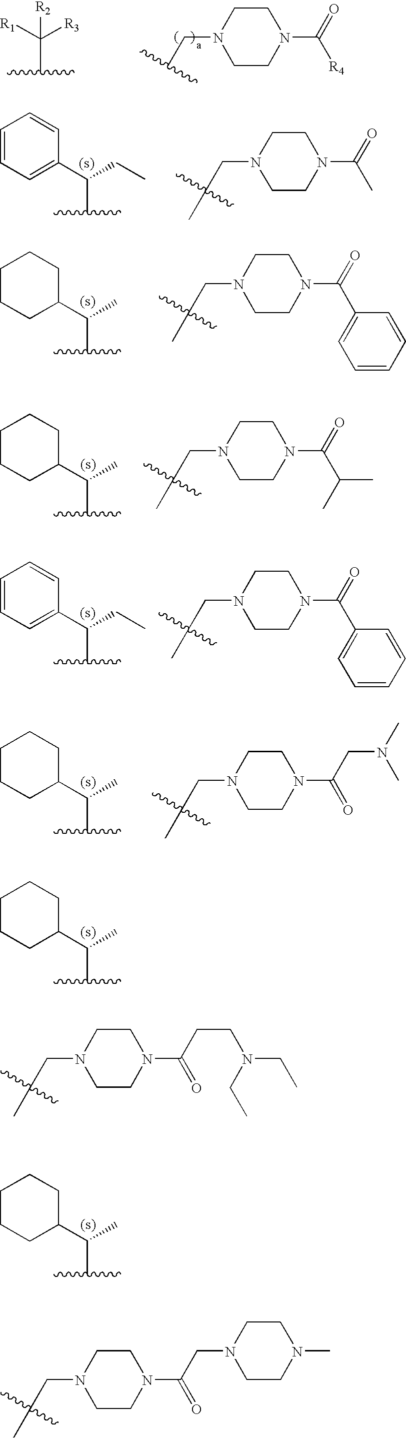

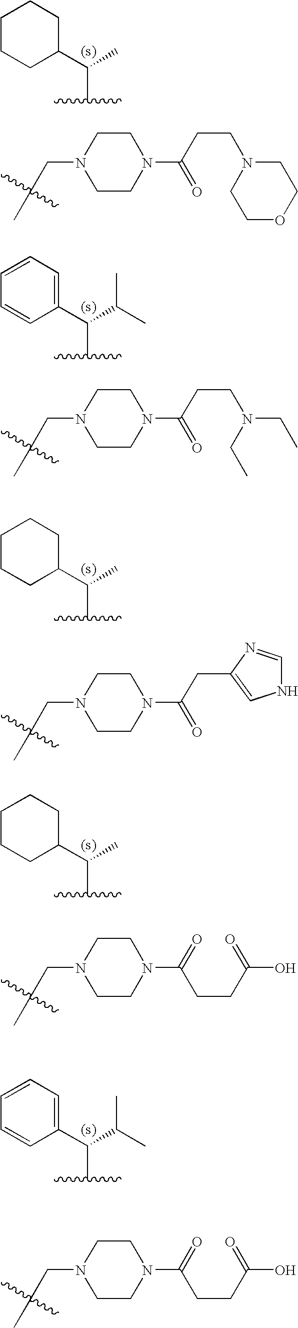

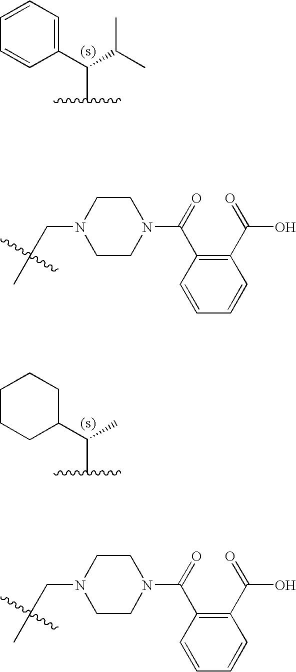

2. A compound as claimed in claim 1, wherein R₃ represents methyl, ethyl or iso-propyl.

3. A compound as claimed in claim 1, wherein R₃ represents methyl.

4. A compound as claimed in claim 1, wherein R₂ represents unsubstituted phenyl or unsubstituted cyclohexyl.

5. A compound as claimed in claim 1, wherein R₁ is hydrogen.

6. A compound as claimed in claim 1, wherein R₅ is unsubstituted phenyl.

7. A compound as claimed in claim 1, wherein each of R₆ and R₇ represents hydrogen.

8. A compound as claimed in claim 1, wherein a is 1, 2 or 3.

9. A compound as claimed in claim 1, wherein a is 1.

10. A compound as claimed in claim 1, wherein R₄ is R₈NR₉R₁₀ and R₈ is a single bond, or methyl, or ethyl.

11. A compound as claimed in claim 1, wherein R₄ is R₈NR₉R₁₀ and each of R₉ and R₁₀ is H.

12. A compound as claimed in claim 1, wherein R₄ is R₈NR₉R₁₀; one of R₉ and R₁₀ is H, and the other of R₉ and R₁₀ is methyl or ethyl or phenyl.

13. A compound as claimed in claim 1, wherein R₄ is R₈NR₉R₁₀ and R₉ and R₁₀ together with the N atom to which they are attached form a saturated heterocyclic ring comprising exactly one N heteroatom.

14. A compound as claimed in claim 1, wherein R₄ is R₁₁R₁₃ or R₁₁R₁₂R₁₃; and R₁₁ is a six-membered heteroaryl ring having one or two N heteroatoms, or a phenyl ring.

15. A compound as claimed in claim 14, wherein said heteroaryl or phenyl ring is ortho-, para- or meta-linked to R₁₂ or R₁₃.

16. A compound as claimed in claim 1, wherein R₄ is R₁₁R₁₃ or R₁₁R₁₂R₁₃; and R₁₁ is cycloalkylalkyl, or alkyl substituted by alkyl or phenyl.

17. A compound as claimed in claim 1, wherein R₄ is R₁₁R₁₂R₁₃, and R₁₂ is methyl or methoxy.

18. A compound as claimed in claim 1, wherein R₄ is R₁₁R₁₃ or R₁₁R₁₂R₁₃; R₁₃ is COO R₁₄; and R₁₄ is H or methyl or ethyl.

19. A compound as claimed in claim 1, wherein a is 1, R₆ is H, R₁ is H, R₅ is unsubstituted phenyl, R₇ is hydrogen, and R₂, R₃ and R₄ are selected from the following combinations:

| R₂ | R₃ | R₄ |
|---|---|---|
| Phenyl | ethyl | 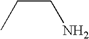 |
| Phenyl | methyl | 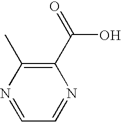 |
| Phenyl | methyl | 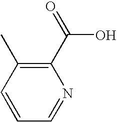 |
| Phenyl | methyl | 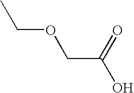 |
| Phenyl | methyl | 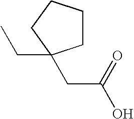 |
| Phenyl | methyl | 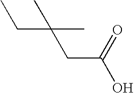 |
| Phenyl | methyl | 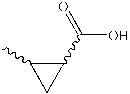 |
| Phenyl | methyl | 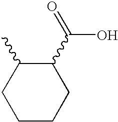 |
| Phenyl | methyl | 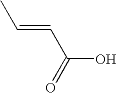 |
| Cyclohexyl | methyl | 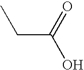 |
| Cyclohexyl | methyl | 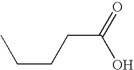 |
| Cyclohexyl | methyl | 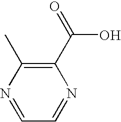 |
| Cyclohexyl | methyl | 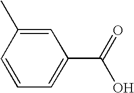 |
| Cyclohexyl | methyl | 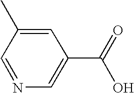 |
| Cyclohexyl | methyl | 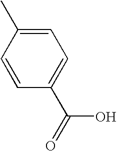 |
| Cyclohexyl | methyl | 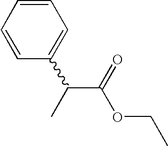 |
| Cyclohexyl | methyl | 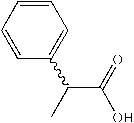 |
| Cyclohexyl | methyl | —H |
| Cyclohexyl | methyl | 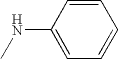 |
| Cyclohexyl | methyl | —NH₂ |
| cyclohexyl | methyl | 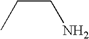 |
| cyclohexyl | methyl | 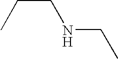 |
| cyclohexyl | methyl | 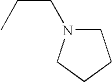 |
| cyclohexyl | methyl | 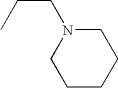 |

20. A process for the preparation of a compound of formula (I) according to claim 1, or a salt thereof and/or a solvate thereof, which process comprises reacting a compound of formula (II) or an active derivative thereof: 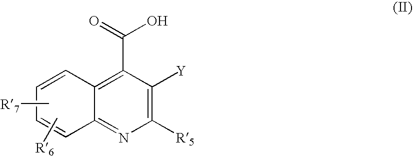
wherein R′₅, R′₆, and R′₇ are R₅, R₆, and R₇ respectively as defined in relation to formula (I) as claimed in claim 1 or a group convertible to R₅, R₆, and R₇ respectively, and Y′ is a group of formula (Y) or a group convertible thereto 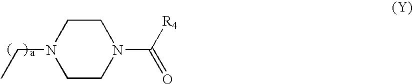
wherein R₄ is defined as in relation to formula (I) as claimed in claim 1, with a compound of formula (III): 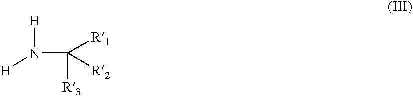
wherein R′₁, R′₂ and R′₃ are R₁, R₂ and R₃ as defined for formula (I) as claimed in claim 1 or a group or atom convertible to R₁, R₂ and R₃ respectively; to form a compound of formula (Ib): 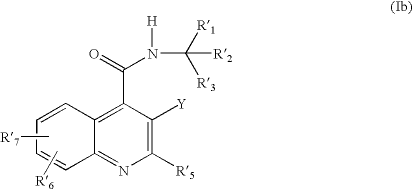
and thereafter carrying out one or more of the following optional steps:
(i) converting any one of R′₁, R′₂, R′₃, R′₅, R′₆, R′₇ and Y′ to R₁, R₂, R₃, R₅, R₆, R₇ and Y respectively as required, to obtain a compound of formula (I) as claimed in claim 1;
(ii) converting a compound of formula (I) as claimed in claim 1 into another compound of formula (I) as claimed in claim 1; and
(iii) preparing a salt of the compound of formula (I) as claimed in claim 1 and/or a solvate thereof.

21. A process for the preparation of a compound of formula (I) according to claim 1, wherein a is 1, or a salt thereof and/or a solvate thereof, which process comprises reacting a compound of formula (T) or an active derivative thereof: 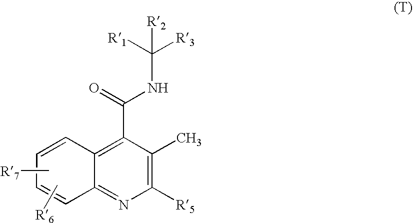
wherein each of R′₁, R′₂, R′₃, R′₅, R′₆, and R′₇ is R₁, R₂, R₃, R₅, R₆, or R₇ respectively as defined in relation to formula (I) or a group convertible to R₁, R₂, R₃, R₅, R₆, or R₇ respectively, providing that R′₂ is not an aromatic group, with a compound of formula (W) 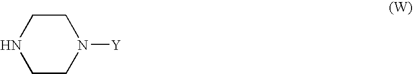
wherein Y is a group COR₄ or a protected form thereof or a group convertible thereto, to form a compound of formula (Ib): 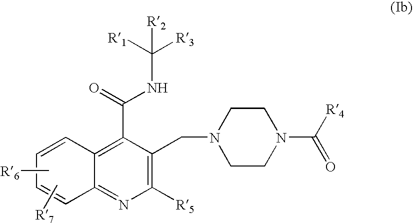
and thereafter carrying out one or more of the following optional steps:
(i) converting any one of R′₁, R′₂, R′₃, R′₄, R′₅, R′₆, and R′₇ to R₁, R₂, R₃, R₄, R₅, R₆, and R₇ respectively as required, to obtain a compound of formula (I) converting a compound of formula (I) into another compound of formula (I); and
(iv) preparing a salt of the compound of formula (I) as claimed in claim 1 and/or a solvate thereof.

22. A pharmaceutical composition comprising a compound of formula (I) according to claim 1, or a pharmaceutically acceptable salt or solvate thereof, and a pharmaceutically acceptable carrier

23. A compound of formula (I) according to claim 1, or a pharmaceutically acceptable salt or solvate thereof, for use as an active therapeutic substance.

24. A compound of formula (I) according to claim 1, or a pharmaceutically acceptable salt or solvate thereof, for the treatment or prophylaxis of the Primary and Secondary Conditions.

25. (canceled)

26. A method for the treatment and/or prophylaxis of the Primary and Secondary Conditions in mammals, particularly humans, which comprises administering to the mammal in need of such treatment and/or prophylaxis an effective, non-toxic pharmaceutically acceptable amount of a compound of formula (I) according to claim 1, or a pharmaceutically acceptable salt or solvate thereof.
